SEQUENCE LISTING (5 sequences)

SEQ ID NO: 1 - protein, 9 aa, Artificial
  Integrin alfa9 beta1 recognition sequence
EDDMMEVPY

SEQ ID NO: 2 - protein, 3567 aa, Mus musculus
MWSRLAFCCWALALVSGWTNFQPVAPSLNFSFRLFPEASPGALGRLAVPPASSEEEAAGSKVERLGRAFRSRVRRLRELSGSLELVFLVDESSSVGQTNFLNELKFVRKLLSDFPVVSTATRVAIVTFSSKNNVVARVDYISTSRAHQHKCALLSREIPAITYRGGGTYTKGAFQQAAQILRHSRENSTKVIFLITDGYSNGGDPRPIAASLRDFGVEIFTFGIWQGNIRELNDMASTPKEEHCYLLHSFEEFEALARRALHEDLPSGSFIQEDMARCSYLCEAGKDCCDRMASCKCGTHTGQFECICEKGYYGKGLQHECTACPSGTYKPEASPGGISTCIPCPDVSHTSPPGSTSPEDCVCREGYQRSGQTCEVVHCPALKPPENGFFIQNTCKNHFNAACGVRCRPGFDLVGSSIHLCQPNGLWSGTESFCRVRTCPHLRQPKHGHISCSTAEMSYNTLCLVTCNEGYRLEGSTRLTCQGNAQWDGPEPRCVERHCATFQKPKGVIISPPSCGKQPARPGMTCQLSCRQGYILSGVREVRCATSGKWSAKVQTAVCKDVEAPQISCPNDIEAKTGEQQDSANVTWQVPTAKDNSGEKVSVHVHPAFTPPYLFPIGDVAITYTATDSSGNQASCTFYIKVIDVEPPVIDWCRSPPPIQVVEKEHPASWDEPQFSDNSGAELVITSSHTQGDMFPHGETVVWYTATDPSGNNRTCDIHIVIKGSPCEVPFTPVNGDFICAQDSAGVNCSLSCKEGYDFTEGSTEKYYCAFEDGIWRPPYSTEWPDCAIKRFANHGFKSFEMLYKTTRCDDMDLFKKFSAAFETTLGNMVPSFCNDADDIDCRLEDLTKKYCIEYNYNYENGFAIGPGGWGAGNRLDYSYDHFLDVVQETPTDVGKARSSRIKRTVPLSDPKIQLIFNITASVPLPEERNDTLELENQQRLIKTLETITNRLKSTLNKEPMYSFQLASETVVADSNSLETEKAFLFCRPGSVLRGRMCVNCPLGTSYSLEHSTCESCLMGSYQDEEGQLECKLCPPRTHTEYLHSRSVSECKAQCKQGTYSSSGLETCESCPLGTYQPEFGSRSCLLCPETTTTVKRGAVDISACGVPCPVGEFSRSGLTPCYPCPRDYYQPNAGKSFCLACPFYGTTTITGATSITDCSSFSSTFSAAEESIVPLVAPGHSQNKYEVSSQVFHECFLNPCHNSGTCQQLGRGYVCLCPPGYTGLKCETDIDECSSLPCLNGGICRDQVGGFTCECSLGYSGQICEENINECISSPCLNKGTCTDGLASYRCTCVKGYMGVHCETDVNECQSSPCLNNAVCKDQVGGFSCKCPPGFLGTRCEKNVDECLSQPCQNGATCKDGANSFRCQCPAGFTGTHCELNINECQSNPCRNQATCVDELNSYSCKCQPGFSGHRCETEQPSGFNLDFEVSGIYGYVLLDGVLPTLHAITCAFWMKSSDVINYGTPISYALEDDKDNTFLLTDYNGWVLYVNGKEKITNCPSVNDGIWHHIAITWTSTGGAWRVYIDGELSDGGTGLSIGKAIPGGGALVLGQEQDKKGEGFNPAESFVGSISQLNLWDYVLSPQQVKLLASSCPEELSRGNVLAWPDFLSGITGKVKVDSSSMFCSDCPSLEGSVPHLRPASGNRKPGSKVSLFCDPGFQMVGNPVQYCLNQGQWTQPLPHCERIRCGLPPALENGFYSAEDFHAGSTVTYQCTSGYYLLGDSRMFCTDNGSWNGISPSCLDVDECAVGSDCSEHASCLNTNGSYVCSCNPPYTGDGKNCAEPVKCKAPENPENGHSSGEIYTVGTAVTFSCDEGHELVGVSTITCLETGEWDRLRPSCEAISCGVPPVPENGGVDGSAFTYGSKVVYRCDKGYTLSGDEESACLASGSWSHSSPVCELVKCSQPEDINNGKYILSGLTYLSIASYSCENGYSLQGPSLLECTASGSWDRAPPSCQLVSCGEPPIVKDAVITGSNFTFGNTVAYTCKEGYTLAGPDTIVCQANGKWNSSNHQCLAVSCDEPPNVDHASPETAHRLFGDTAFYYCADGYSLADNSQLICNAQGNWVPPAGQAVPRCIAHFCEKPPSVSYSILESVSKAKFAAGSVVSFKCMEGFVLNTSAKIECLRGGEWSPSPLSVQCIPVRCGEPPSIANGYPSGTNYSFGAVVAYSCHKGFYIKGEKKSTCEATGQWSKPTPTCHPVSCNEPPKVENGFLEHTTGRTFESEARFQCNPGYKAAGSPVFVCQANRHWHSDAPLSCTPLNCGKPPPIQNGFLKGESFEVGSKVQFVCNEGYELVGDNSWTCQKSGKWSKKPSPKCVPTKCAEPPLLENQLVLKELASEVGVMTISCKEGHALQGPSVLKCLPSGQWNGSFPICKMVLCPSPPLIPFGVPASSGALHFGSTVKYLCVDGFFLRGSPTILCQADSTWSSPLPECVPVECPQPEEILNGIIHVQGLAYLSTTLYTCKPGFELVGNATTLCGENGQWLGGKPMCKPIECPEPKEILNGQFSSVSFQYGQTITYFCDRGFRLEGPKSLTCLETGDWDMDPPSCDAIHCSDPQPIENGFVEGADYRYGAMIIYSCFPGFQVLGHAMQTCEESGWSSSSPTCVPIDCGLPPHIDFGDCTKVRDGQGHFDQEDDMMEVPYLAHPQHLEATAKALENTKESPASHASHFLYGTMVSYSCEPGYELLGIPVLICQEDGTWNGTAPSCISIECDLPVAPENGFLHFTQTTMGSAAQYSCKPGHILEGSHLRLCLQNKQWSGTVPRCEAISCSKPNPLWNGSIKGDDYSYLGVLYYECDSGYILNGSKKRTCQENRDWDGHEPMCIPVDCGSPPVPTNGRVKGEEYTFQKEITYSCREGFILEGARSRICLTNGSWSGATPSCMPVRCPAPPQVPNGVADGLDYGFKKEVAFHCLEGYVLQGAPRLTCQSNGTWDAEVPVCKPATCGPPADLPQGFPNGFSFYHGGHIQYQCFTGYKLHGNPSRRCLPNGSWSGSSPSCLPCRCSTPIIQQGTINATDLGCGKTVQIECFKGFKLLGLSEITCDANGQWSDVPLCEHAQCGPLPTIPNAIVLEGSLSEDNVVTYSCRPGYTMQGSSDLICTEKAIWSQPYPTCEPLSCGPPPTVANAVATGEAHTYESKVKLRCLEGYVMDSDTDTFTCQQDGHWVPERITCSPKKCPVPSNMTRIRFHGDDFQVNRQVSVSCAEGFTHEGVNWSTCQPDGTWEPPFSDESCIPVVCGHPESPAHGSVVGNKHSFGSTIVYQCDPGYKLEGNRERICQENRQWSGEVAVCRENRCETPAEFPNGKAVLENTTSGPSLLFSCHRGYTLEGSPEAHCTANGTWNHLTPLCKPNPCPVPFVIPENAVLSEKEFYVDQNVSIKCREGFLLKGNGVITCSPDETWTHTNARCEKISCGPPSHVENAIARGVYYQYGDMITYSCYSGYMLEGSLRSVCLENGTWTPSPVCRAVCRFPCQNGGVCQRPNACSCPDGWMGRLCEEPICILPCLNGGRCVAPYQCDCPTGWTGSRCHTATCQSPCLNGGKCIRPNRCHCLSAWTGHDCSRKRRAGL

SEQ ID NO: 3 - DNA, 11275 bp, Mus musculus
atagccgagagtcagaggagcacatccctccagtccctcccgagtccccgagctgccagaggagtctggatcgtgtccccagtgtcacatgcaaggacgctgaggttcgcggttgctaccccgggtcccctccgcttagtccgggaaccttggcgcctctctgcgcgctcggggactgtcgccttgcactccccggggccaccgctcggtccccagcgggatgtggtcgcgcctggccttttgttgctgggctctggcactggtgtcaggctggaccaacttccagcccgtggccccttcgctcaacttcagcttccgcctgttccccgaggcctctccgggggctctgggcagactggcggtacctcccgcgtccagtgaggaggaggcagcagggagcaaagtggagcgcctgggccgcgcgttccggagccgcgtgcggcgactgcgggagctcagcggcagcctggagctcgtcttcctggtggacgagtcgtccagcgtgggccaaaccaacttcctcaacgagctcaagttcgtgcgcaagctgctgtccgacttccccgtggtgtccacggccacgcgtgtggccatcgtcaccttctcatccaagaacaacgtggtggcgcgcgtggattacatctccaccagccgcgcgcaccaacacaagtgcgcgctactcagccgcgagatcccggccatcacctaccgcggtggtggcacctataccaagggcgccttccagcaagccgcgcaaatccttcgtcactctagagaaaactccaccaaagtcatatttctcatcaccgacggctattccaatggcggagaccccagacctattgcagcatcgcttcgggatttcggagtggagatcttcacgttcgggatttggcaggggaatatccgggaactgaatgacatggcttccaccccgaaggaagaacattgttacctgctccacagttttgaagaatttgaggctttagctcgcagggcgttgcatgaagatctaccttctgggagttttatccaagaggatatggcccgctgctcttatctctgtgaggctgggaaagactgctgtgacagaatggccagctgcaaatgtgggacacacacgggtcaatttgaatgcatctgtgagaagggctattacgggaaaggtctgcagcatgagtgcacagcttgcccatcagggacatataagccggaagcttctccaggaggaatcagcacctgcatcccatgtcctgacgtaagccacacctccccacctggaagcacttcccctgaagactgcgtgtgccgagagggataccagagatctggccagacctgtgaggttgtccactgtcctgccctgaagcctcctgaaaatggtttttttatacaaaacacttgcaaaaaccacttcaatgccgcctgtggggtccgatgtcgcccgggctttgaccttgtgggaagcagcatccatttgtgtcaacccaatggtttgtggtctgggacagaaagcttctgcagagtgagaacgtgcccccacctccgacagcccaaacacggccacatcagctgctccactgcggaaatgtcctacaacaccctgtgtttggttacctgcaatgaaggatacagattagaaggcagcactaggcttacctgtcaaggaaatgcccagtgggatggcccagagccccggtgtgtagaacgccattgtgccaccttccagaagcccaaaggcgtcatcatttctccacccagctgcggcaagcagcctgccaggcctgggatgacctgtcagctaagctgccgccagggatacattttatccggggtcagagaagtgagatgtgccacatctgggaagtggagtgccaaagttcagacagctgtgtgcaaagatgtggaggctccacaaatcagctgtccaaatgacattgaggcaaagactggggagcagcaggactctgctaatgtcacctggcaagtcccaacagctaaagacaactctggtgaaaaggtgtcagtccacgtccacccagcctttaccccaccttacctcttcccaattggagacgtggccatcacctacacggcaaccgactcatccggtaaccaagccagctgcactttctacattaaggtcattgatgtggaaccgcctgtcatagattggtgccgatctccacctccaatccaggtcgtagagaaggagcaccctgcaagctgggatgagcctcagttctcagacaactccggggctgaattggtcattaccagcagtcacacacaaggcgacatgtttcctcatggggaaacggtggtgtggtacacagccactgacccctcaggcaacaacaggacctgtgacatccacattgtcataaaaggttctccctgtgaggtccccttcacccctgtaaacggggactttatctgtgcccaggatagtgctggagttaactgtagcctgagctgcaaggagggctatgatttcacagaagggtcaactgagaagtactactgtgcttttgaagatggtatctggagaccaccatactctacagaatggccagactgtgctataaaacgttttgcaaaccatggtttcaagtcctttgaaatgctatacaaaaccactcgctgtgatgacatggatctgtttaagaagttttctgcagcatttgagactaccctggggaacatggtcccgtccttttgtaacgatgctgatgacattgactgcagactggaggacctgaccaaaaaatactgcatcgagtataattacaactatgaaaatggctttgcaattggaccaggaggctggggtgcaggcaacaggctggattattcctacgatcacttcctggatgttgtacaggaaacacccaccgatgtgggcaaggccagatcgtcacggattaaaagaactgtcccattgtctgaccccaaaattcagctaatttttaacatcacagctagcgtgccactcccagaggaaagaaacgatacccttgaattggagaatcagcagcgactcattaagacattggaaacaatcaccaatcgcctgaaaagcaccttgaataaagagcccatgtattctttccagctcgcctcggaaacagtggtggctgacagcaattccctcgaaacagaaaaggcttttctcttctgcagaccaggctctgtgctgagggggcgcatgtgtgtcaactgccccctgggaacctcttactctctggagcattccacctgtgaaagctgcctcatgggatcctaccaagatgaagaagggcagctggaatgcaagctctgtcccccaaggactcacacggaatacctccattcaagaagcgtctctgaatgcaaagctcagtgtaagcaaggcacctactcttccagtgggctggagacctgcgaatcgtgtccgctgggtacttatcaaccggaatttggatcccggagctgcctcctatgcccagaaaccaccacaacggtgaaaagaggagccgtggacatctctgcttgtggagtgccctgcccagtaggagaattctcccgttctgggctaacaccctgctacccttgccctcgagactattaccaacccaatgcagggaagtccttctgcctcgcttgtcccttttatggaactacaaccatcactggcgccacgtccatcacagactgctcaagttttagctctactttctcagcagcagaagaaagcatagtgcccctcgtggcccctggacattcccagaacaagtacgaagtcagcagtcaggtctttcacgaatgcttcttaaacccctgccacaacagtggaacctgccaacagcttgggcgtggttatgtctgtctctgcccacctggatacacaggcttaaagtgtgaaacagatattgatgaatgcagctctctgccttgcctcaatggtggaatttgtagagaccaagttgggggattcacgtgcgaatgttcattgggctattcaggtcaaatatgtgaagaaaatataaatgagtgtatctccagcccttgcttaaataaaggaacctgcactgacggcttggcaagctaccgctgtacctgtgtgaaaggatacatgggtgtgcactgtgaaacagacgtcaatgaatgccagtcaagcccctgcttaaacaacgcagtttgtaaagaccaagttggggggttctcatgcaaatgcccacccggatttttgggtactcggtgtgaaaaaaatgtggatgagtgtctcagtcagccatgccaaaatggagccacttgtaaggatggtgccaacagcttcaggtgtcaatgtccagcaggcttcacagggacacactgtgaactgaacatcaacgagtgtcagtccaacccatgtaggaaccaggccacctgtgtggatgaactaaactcatacagttgtaaatgtcagccaggattttcaggccacaggtgtgagacagaacagccttccggttttaacctggattttgaagtttctggcatctacgggtacgtcctgctagatggagtgctgccaaccctccatgccataacctgcgcattctggatgaaatcctctgatgtcatcaactacgggacgcccatctcctatgcacttgaggatgacaaagacaacaccttcctcctgactgattacaacggctgggttctttatgtgaatggaaaggaaaagatcaccaactgcccctccgtaaatgatggcatttggcatcatattgcaatcacatggacaagtactggtggagcctggagggtctatatagatggggaattatctgacggtggtactggcctctccattggcaaagccatacctggtggcggtgcattagttcttgggcaagagcaagacaaaaaaggagaggggttcaacccggctgagtcttttgtgggctccataagccagctcaacctctgggactatgtcctgtctccacagcaggtgaagttgctggccagctcctgcccagaggaactgagtcggggaaacgtgttagcatggcccgatttcctgtcgggaatcacggggaaggtgaaggttgattccagcagcatgttctgctctgattgtccgtctttagaaggatccgtgcctcacctgagacctgcatcaggaaatcgaaagccaggctccaaagtcagtctgttctgtgatccgggcttccagatggttgggaatcctgtgcagtattgtctgaaccaagggcagtggacacaaccactcccccactgtgaacgcattcgctgtgggctgcctcccgccttggagaatggcttctactcagccgaggacttccatgcgggcagcacggtgacctatcagtgcaccagtggctactacctgctgggtgattcccgaatgttctgcacagacaacgggagctggaacggcatttcaccatcctgtctcgatgtcgatgagtgtgcagtcggctcggactgtagtgagcacgcctcctgcctgaacaccaacggatcctacgtatgttcctgtaacccaccatacacgggagatgggaaaaactgtgcagaacctgtaaaatgtaaggctccagaaaatccagaaaatggccactcttctggtgagatttacaccgtgggtactgcagtcacattttcctgtgacgaagggcacgagctggtgggagtgagcaccatcacgtgtttggagactggcgagtgggatcgcctcaggccgtcctgtgaagccatttcctgtggtgtcccacctgttcctgaaaatggtggtgttgacgggtcggcattcacatacggcagtaaggtggtgtacaggtgtgataaaggatatactttgtctggggatgaagagtcagcatgccttgctagtggttcctggagtcattcctctcctgtgtgcgagctagtgaagtgttcccagcctgaggacataaataacggcaaatacatcttaagtgggctcacctacctttctattgcatcgtactcctgtgagaacggatacagtttacagggcccatccctccttgaatgcacagcttccggcagctgggacagagcgccacctagctgtcaacttgtctcctgcggagagcctccaatcgtcaaagatgctgtcatcactgggagcaacttcacttttgggaacacagttgcttacacatgcaaagagggctacacccttgctgggcctgacaccatcgtatgccaggccaacggcaaatggaattcaagtaaccaccagtgcctggctgtctcctgtgacgagccccccaatgtggaccacgcctctccagagactgctcacaggctctttggagacaccgcgttttactactgtgcggatggttacagcctggctgataattcccagctcatctgcaatgcccaggggaactgggttccccccgcgggccaggctgtgccgcgctgcatagctcacttctgtgaaaaacccccatctgtttcctacagcatcttggaatctgtgagcaaagcaaagtttgcagctggctcggtagtgagcttcaagtgcatggagggttttgtgctgaacacctcagcgaagattgaatgcctgagaggtggagagtggagcccttctcccctctcggtccagtgcatcccggtgcgatgcggagagcctccaagcatcgcaaatggctacccgagtgggacaaactacagttttggggccgtggtggcctacagctgccacaagggattctatatcaagggggagaagaagagcacgtgtgaggccacaggacagtggagtaaacccacgcccacctgccatcctgtgtcctgtaacgagccacctaaggttgagaacggcttcctggagcacaccactggcaggacctttgagagcgaagcaaggttccagtgcaacccaggctataaggcagccggaagtcctgtgtttgtttgccaagccaatcgccactggcacagcgacgcccctctgtcctgcacccctctcaactgtgggaaaccccctcccattcagaatggctttttgaaaggagaaagctttgaagtagggtccaaggttcagtttgtctgtaatgagggatatgagctcgttggtgataattcttggacttgccagaaatctggcaaatggagtaagaagccaagcccgaagtgtgtccccaccaagtgtgcagagcctcctctcttagaaaaccagctcgtattgaaggaattagcttccgaggtaggagtgatgaccatttcctgtaaagaggggcatgccttgcaaggcccctctgtcctgaagtgcttgccatccgggcaatggaatggttcctttcctatttgtaagatggtcctttgtccctcgcctcccttgattcccttcggcgtccctgcgtcttccggtgctcttcattttggcagtactgtcaagtatctgtgtgtcgacgggtttttcttaagaggcagtccaaccatcctctgccaggctgatagcacctggagttctccattgcccgaatgcgttccggtagaatgtccccaacctgaggagatcctcaacggtatcatccacgtacaagggcttgcctatctcagcaccacgctctacacctgcaagccaggctttgagttagtgggcaatgctaccaccctctgtggggaaaatggccagtggctcggaggaaaaccaatgtgcaaacccattgaatgcccagagcccaaggagattttaaatggccaattctcttccgtgagctttcagtatggacaaaccatcacatacttttgtgaccggggcttccggctcgaaggtcccaaatccctgacctgtttagagacaggtgactgggatatggatcccccctcttgtgatgccatccactgcagtgacccacagcccattgaaaatggtttcgtagaaggtgcggattacagatacggtgccatgatcatctatagctgcttccctgggtttcaggtgcttggtcatgccatgcagacctgtgaagagtcgggatggtcaagctccagcccaacctgtgtacccatagactgcggtctccctcctcacatagactttggtgactgtactaaagtcagagatggccagggacattttgatcaagaagatgacatgatggaagtcccatatctggctcaccctcaacatttggaagcaacagctaaggccttggaaaatacaaaggagtcgcctgcctcacatgcatcccacttcctctatggcacgatggtttcctacagctgcgagcctggttatgaactgctgggaatccctgtgctgatctgccaggaagatggtacgtggaatggtaccgcaccctcttgcatttccattgaatgtgatttgcctgttgctcccgaaaatggctttttacatttcacacagacgactatgggcagtgctgcacaatatagctgcaagccggggcacattctagaaggctcccacttaagactctgtctgcagaataagcagtggagtggcactgttccacgctgtgaagccatctcatgcagtaagccaaacccactctggaatggatccatcaaaggagatgactactcctacctgggtgtgttatactacgagtgtgactctggctatattctcaatggctctaagaagaggacatgccaagaaaatagagattgggatgggcatgagcccatgtgtattcctgtagactgtggctcacccccagtccccaccaatggccgagtgaagggagaagaatacacattccaaaaggagattacatactcttgccgtgaagggttcatactggaaggagccaggagtcgtatctgtcttaccaatggaagttggagtggtgccactcccagctgcatgcctgttagatgtcctgccccaccacaggtgccaaatggggtggcagatggcctagactatgggttcaagaaggaagtagcgttccactgtctagagggctatgtgctgcagggggctccaagactcacctgtcagtccaatgggacttgggatgcagaagtccctgtctgtaaaccagctacctgtggtcctcctgccgaccttccccagggcttccctaatggcttttctttttatcatgggggccacatacagtatcagtgttttactggttataagcttcatggaaacccatcaagaagatgccttcccaatggttcctggagcggcagctcgccatcctgcctaccttgcaggtgttccacacccatcattcaacagggaaccatcaacgcaactgatttgggatgtggaaagacggtccagattgagtgcttcaaaggcttcaagctgcttggactttctgaaatcacctgtgatgccaatggccaatggtctgacgtcccactgtgtgagcacgctcagtgcgggcctctcccaaccatacccaacgcaattgtccttgagggcagcctttcggaggacaatgtggtaacttacagctgcagacctggctacaccatgcaaggtagttcagatctgatttgtacggaaaaagcgatatggagccagccttacccaacgtgtgaacccctgtcctgtggacccccaccaactgtagccaatgcagtggcaacaggagaggctcatacctatgaaagcaaagtgaaactcaggtgtctggaagggtatgtgatggattcggatacagatacattcacctgccagcaagatggccattgggtccctgaaagaatcacctgcagtcctaaaaaatgccctgtgccatccaacatgacacgcatacgttttcacggagatgacttccaggtgaacagacaagtttctgtgtcatgtgcagaagggtttacccacgaaggagtgaactggtcaacatgccagcccgacggtacatgggagccaccattttctgatgaatcctgtatcccagttgtttgtgggcatcctgaaagcccagcgcatggctccgtggttggcaataagcacagctttggaagcaccattgtttaccagtgtgaccctggctacaaattagaggggaacagggaacgaatctgccaggagaacagacagtggagtggagaggtggcagtgtgcagagagaacagatgtgagactccagctgagtttcccaatgggaaggctgtcttggaaaacaccacatctggacccagccttctgttttcctgtcacagaggctacaccctggaagggtcccccgaggcacactgcactgcaaatggaacctggaatcacctgactcccctctgcaaaccaaatccatgccctgtcccttttgtgattcctgagaacgccgtcctttctgaaaaagagttttatgtcgaccagaatgtatctatcaagtgcagggaaggcttcctgctcaaaggcaatggtgtcatcacgtgcagccctgacgagacatggacgcacaccaatgccagatgtgaaaaaatctcctgtggtcctccaagtcacgtagagaatgcaattgctcgaggagtgtattaccagtatggggacatgatcacctactcctgttacagtggctacatgctagaaggttccctccggagtgtttgcctagaaaatggaacatggacaccatctcctgtttgcagagctgtctgtcggttcccatgtcagaatggaggtgtctgtcaacgtccaaatgcttgctcatgcccagacggctggatgggacgtctctgtgaagagccaatatgcatactcccctgtttgaatggtgggcgctgtgtggccccttatcagtgtgactgccccacaggctggactgggtcccgctgtcatacagctacttgtcagtccccctgcttaaatggcgggaaatgcataagaccaaaccgatgccattgtctctcagcctggacaggacatgattgttccaggaaaaggagagccgggctttgatctcatgccccaccccctctccctaagcagcatcatctccttccggtagctcctgggactcccaccaagaaagaccaacgcggtgctggggcttgtttggttttataagcttgaggttgactttttaattttgtgatctattttgttaaatttttttgtgacgtcctttcttacatgtgtgcgttgtttaaatatgcttgcattttctatataaaatttatattaaacggacgcacttcatcctcaccagatgtacatactctgctgtctgctgggaaagcccctggaatacatttttattcaattacttaaagatgactttccattaaaatatattttgctactaaataa

SEQ ID NO: 4 - protein, 3571 aa, Homo sapiens
MWPRLAFCCWGLALVSGWATFQQMSPSRNFSFRLFPETAPGAPGSIPAPPAPGDEAAGSRVERLGQAFRRRVRLLRELSERLELVFLVDDSSSVGEVNFRSELMFVRKLLSDFPVVPTATRVAIVTFSSKNYVVPRVDYISTRRARQHKCALLLQEIPAISYRGGGTYTKGAFQQAAQILLHARENSTKVVFLITDGYSNGGDPRPIAASLRDSGVEIFTFGIWQGNIRELNDMASTPKEEHCYLLHSFEEFEALARRALHEDLPSGSFIQDDMVHCSYLCDEGKDCCDRMGSCKCGTHTGHFECICEKGYYGKGLQYECTACPSGTYKPEGSPGGISSCIPCPDENHTSPPGSTSPEDCVCREGYRASGQTCELVHCPALKPPENGYFIQNTCNNHFNAACGVRCHPGFDLVGSSIILCLPNGLWSGSESYCRVRTCPHLRQPKHGHISCSTREMLYKTTCLVACDEGYRLEGSDKLTCQGNSQWDGPEPRCVERHCSTFQMPKDVIISPHNCGKQPAKFGTICYVSCRQGFILSGVKEMLRCTTSGKWNVGVQAAVCKDVEAPQINCPKDIEAKTLEQQDSANVTWQIPTAKDNSGEKVSVHVHPAFTPPYLFPIGDVAIVYTATDLSGNQASCIFHIKVIDAEPPVIDWCRSPPPVQVSEKVHAASWDEPQFSDNSGAELVITRSHTQGDLFPQGETIVQYTATDPSGNNRTCDIHIVIKGSPCEIPFTPVNGDFICTPDNTGVNCTLTCLEGYDFTEGSTDKYYCAYEDGVWKPTYTTEWPDCAKKRFANHGFKSFEMFYKAARCDDTDLMKKFSEAFETTLGKMVPSFCSDAEDIDCRLEENLTKKYCLEYNYDYENGFAIGPGGWGAANRLDYSYDDFLDTVQETATSIGNAKSSRIKRSAPLSDYKIKLIFNITASVPLPDERNDTLEWENQQRLLQTLETITNKLKRTLNKDPMYSFQLASEILIADSNSLETKKASPFCRPGSVLRGRMCVNCPLGTYYNLEHFTCESCRIGSYQDEEGQLECKLCPSGMYTEYIHSRNISDCKAQCKQGTYSYSGLETCESCPLGTYQPKFGSRSCLSCPENTSTVKRGAVNISACGVPCPEGKFSRSGLMPCHPCPRDYYQPNAGKAFCLACPFYGTTPFAGSRSITECSSFSSTFSAAEESVVPPASLGHIKKRHEISSQVFHECFFNPCHNSGTCQQLGRGYVCLCPLGYTGLKCETDIDECSPLPCLNNGVCKDLVGEFICECPSGYTGQRCEENINECSSSPCLNKGICVDGVAGYRCTCVKGFVGLHCETEVNECQSNPCLNNAVCEDQVGGFLCKCPPGFLGTRCGKNVDECLSQPCKNGATCKDGANSFRCLCAAGFTGSHCELNINECQSNPCRNQATCVDELNSYSCKCQPGFSGKRCETEQSTGFNLDFEVSGIYGYVMLDGMLPSLHALTCTFWMKSSDDMNYGTPISYAVDNGSDNTLLLTDYNGWVLYVNGREKITNCPSVNDGRWHHIAITWTSANGIWKVYIDGKLSDGGAGLSVGLPIPGGGALVLGQEQDKKGEGFSPAESFVGSISQLNLWDYVLSPQQVKSLATSCPEELSKGNVLAWPDFLSGIVGKVKIDSKSIFCSDCPRLGGSVPHLRTASEDLKPGSKVNLFCDPGFQLVGNPVQYCLNQGQWTQPLPHCERISCGVPPPLENGFHSADDFYAGSTVTYQCNNGYYLLGDSRMFCTDNGSWNGVSPSCLDVDECAVGSDCSEHASCLNVDGSYICSCVPPYTGDGKNCAEPIKCKAPGNPENGHSSGEIYTVGAEVTFSCQEGYQLMGVTKITCLESGEWNHLIPYCKAVSCGKPAIPENGCIEELAFTFGSKVTYRCNKGYTLAGDKESSCLANSSWSHSPPVCEPVKCSSPENINNGKYILSGLTYLSTASYSCDTGYSLQGPSIIECTASGIWDRAPPACHLVFCGEPPAIKDAVITGNNFTFRNTVTYTCKEGYTLAGLDTIECLADGKWSRSDQQCLAVSCDEPPIVDHASPETAHRLFGDIAFYYCSDGYSLADNSQLLCNAQGKWVPPEGQDMPRCIAHFCEKPPSVSYSILESVSKAKFAAGSVVSFKCMEGFVLNTSAKIECMRGGQWNPSPMSIQCIPVRCGEPPSIMNGYASGSNYSFGAMVAYSCNKGFYIKGEKKSTCEATGQWSSPIPTCHPVSCGEPPKVENGFLEHTTGRIFESEVRYQCNPGYKSVGSPVFVCQANRHWHSESPLMCVPLDCGKPPPIQNGFMKGENFEVGSKVQFFCNEGYELVGDSSWTCQKSGKWNKKSNPKCMPAKCPEPPLLENQLVLKELTTEVGVVTFSCKEGHVLQGPSVLKCLPSQQWNDSFPVCKIVLCTPPPLISFGVPIPSSALHFGSTVKYSCVGGFFLRGNSTTLCQPDGTWSSPLPECVPVECPQPEEIPNGIIDVQGLAYLSTALYTCKPGFELVGNTTTLCGENGHWLGGKPTCKAIECLKPKEILNGKFSYTDLHYGQTVTYSCNRGFRLEGPSALTCLETGDWDVDAPSCNAIHCDSPQPIENGFVEGADYSYGAIIIYSCFPGFQVAGHAMQTCEESGWSSSIPTCMPIDCGLPPHIDFGDCTKLKDDQGYFEQEDDMMEVPYVTPHPPYHLGAVAKTWENTKESPATHSSNFLYGTMVSYTCNPGYELLGNPVLICQEDGTWNGSAPSCISIECDLPTAPENGFLRFTETSMGSAVQYSCKPGHILAGSDLRLCLENRKWSGASPRCEAISCKKPNPVMNGSIKGSNYTYLSTLYYECDPGYVLNGTERRTCQDDKNWDEDEPICIPVDCSSPPVSANGQVRGDEYTFQKEIEYTCNEGFLLEGARSRVCLANGSWSGATPDCVPVRCATPPQLANGVTEGLDYGFMKEVTFHCHEGYILHGAPKLTCQSDGNWDAEIPLCKPVNCGPPEDLAHGFPNGFSFIHGGHIQYQCFPGYKLHGNSSRRCLSNGSWSGSSPSCLPCRCSTPVIEYGTVNGTDFDCGKAARIQCFKGFKLLGLSEITCEADGQWSSGFPHCEHTSCGSLPMIPNAFISETSSWKENVITYSCRSGYVIQGSSDLICTEKGVWSQPYPVCEPLSCGSPPSVANAVATGEAHTYESEVKLRCLEGYTMDTDTDTFTCQKDGRWFPERISCSPKKCPLPENITHILVHGDDFSVNRQVSVSCAEGYTFEGVNISVCQLDGTWEPPFSDESCSPVSCGKPESPEHGFVVGSKYTFESTIIYQCEPGYELEGNRERVCQENRQWSGGVAICKETRCETPLEFLNGKADIENRTTGPNVVYSCNRGYSLEGPSEAHCTENGTWSHPVPLCKPNPCPVPFVIPENALLSEKEFYVDQNVSIKCREGFLLQGHGIITCNPDETWTQTSAKCEKISCGPPAHVENAIARGVHYQYGDMITYSCYSGYMLEGFLRSVCLENGTWTSPPICRAVCRFPCQNGGICQRPNACSCPEGWMGRLCEEPICILPCLNGGRCVAPYQCDCPPGWTGSRCHTAVCQSPCLNGGKCVRPNRCHCLSSWTGHNCSRKRRTGF

SEQ ID NO: 5 - DNA, 12356 bp, Homo sapiens
agggagagaaggagggaagcgggagatttttcttgactgccccctttccttcaaacattttataggcttcagggagagagaggaggaggagagagggaagaaaaaaagaggagagcgagaggggtagagagcgcgcgccgttccctccggagttcccgagctgctgaggagtctggattgtgtctgtccccagtgtcagatgaaagggcgctgaggctcttggccgctgccccgcgcccagctccgcgcacgcccctctgcgagtccggccgcccagcgcctcttcccgcccgagccgccgcctgcgctccggggcagccgctctgtctccagcgcgatgtggcctcgcctggccttttgttgctggggtctggcgctcgtttcgggctgggcgacctttcagcagatgtccccgtcgcgcaatttcagcttccgcctcttccccgagaccgcgcccggggcccccgggagtatccccgcgccgcccgctcctggcgacgaagcggcggggagcagagtggagcggctgggccaggcgttccggcgacgcgtgcggctgctgcgggagctcagcgagcgcctggagcttgtcttcctggtggatgattcgtccagcgtgggcgaagtcaacttccgcagcgagctcatgttcgtccgcaagctgctgtccgacttccccgtggtgcccacggccacgcgcgtggccatcgtgaccttctcgtccaagaactacgtggtgccgcgcgtcgattacatctccacccgccgcgcgcgccagcacaagtgcgcgctgctcctccaagagatccctgccatctcctaccgaggtggcggcacctacaccaagggcgccttccagcaagccgcgcaaattcttcttcatgctagagaaaactcaacaaaagttgtatttctcatcactgatggatattccaatgggggagaccctagaccaattgcagcgtcactgcgagattcaggagtggagatcttcacttttggcatatggcaagggaacattcgagagctgaatgacatggcttccaccccaaaggaggagcactgttacctgctacacagttttgaagaatttgaggctttagctcgccgggcattgcatgaagatctaccttctgggagttttattcaagatgatatggtccactgctcatatctttgtgatgaaggcaaggactgctgtgaccgaatgggaagctgcaaatgtgggacacacacaggccattttgagtgcatctgtgaaaaggggtattacgggaaaggtctgcagtatgaatgcacagcttgcccatcggggacatacaaacctgaaggctcaccaggaggaatcagcagttgcattccatgtcctgatgaaaatcacacctctccacctggaagcacatcccctgaagactgtgtctgcagagagggatacagggcatctggccagacctgtgaacttgtccactgccctgccctgaagcctcccgaaaatggttactttatccaaaacacttgcaacaaccacttcaatgcagcctgtggggtccgatgtcaccctggatttgatcttgtgggaagcagcatcatcttatgtctacccaatggtttgtggtccggttcagagagctactgcagagtaagaacatgtcctcatctccgccagccgaaacatggccacatcagctgttctacaagggaaatgttatataagacaacatgtttggttgcctgtgatgaagggtacagactagaaggcagtgataagcttacttgtcaaggaaacagccagtgggatgggccagaaccccggtgtgtggagcgccactgttccacctttcagatgcccaaagatgtcatcatatccccccacaactgtggcaagcagccagccaaatttgggacgatctgctatgtaagttgccgccaagggttcattttatctggagtcaaagaaatgctgagatgtaccacttctggaaaatggaatgtcggagttcaggcagctgtgtgtaaagacgtggaggctcctcaaatcaactgtcctaaggacatagaggctaagactctggaacagcaagattctgccaatgttacctggcagattccaacagctaaagacaactctggtgaaaaggtgtcagtccacgttcatccagctttcaccccaccttaccttttcccaattggagatgttgctatcgtatacacggcaactgacctatccggcaaccaggccagctgcattttccatatcaaggttattgatgcagaaccacctgtcatagactggtgcagatctccacctcccgtccaggtctcggagaaggtacatgccgcaagctgggatgagcctcagttctcagacaactcaggggctgaattggtcattaccagaagtcatacacaaggagaccttttccctcaaggggagactatagtacagtatacagccactgacccctcaggcaataacaggacatgtgatatccatattgtcataaaaggttctccctgtgaaattccattcacacctgtaaatggggattttatatgcactccagataatactggagtcaactgtacattaacttgcttggagggctatgatttcacagaagggtctactgacaagtattattgtgcttatgaagatggcgtctggaaaccaacatataccactgaatggccagactgtgccaaaaaacgttttgcaaaccacgggttcaagtcctttgagatgttctacaaagcagctcgttgtgatgacacagatctgatgaagaagttttctgaagcatttgagacgaccctgggaaaaatggtcccatcattttgtagtgatgcagaggacattgactgcagactggaggagaacctgaccaaaaaatattgcctagaatataattatgactatgaaaatggctttgcaattggaccaggtggctggggtgcagctaataggctggattactcttacgatgacttcctggacactgtgcaagaaacagccacaagcatcggcaatgccaagtcctcacggattaaaagaagtgccccattatctgactataaaattaagttaatttttaacatcacagctagtgtgccattacccgatgaaagaaatgatacccttgaatgggaaaatcagcaacgactccttcagacattggaaactatcacaaataaactgaaaaggactctcaacaaagaccccatgtattcctttcagcttgcatcagaaatacttatagccgacagcaattcattagaaacaaaaaaggcttcccccttctgcagaccaggctcagtgctgagagggcgtatgtgtgtcaattgccctttgggaacctattataatctggaacatttcacctgtgaaagctgccggatcggatcctatcaagatgaagaagggcaacttgagtgcaagctttgcccctctgggatgtacacggaatatatccattcaagaaacatctctgattgtaaagctcagtgtaaacaaggcacctactcatacagtggacttgagacttgtgaatcgtgtccactgggcacttatcagccaaaatttggttcccggagctgcctctcgtgtccagaaaacacctcaactgtgaaaagaggagccgtgaacatttctgcatgtggagttccttgtccagaaggaaaattctcgcgttctgggttaatgccctgtcacccatgtcctcgtgactattaccaacctaatgcagggaaggccttctgcctggcctgtcccttttatggaactaccccattcgctggttccagatccatcacagaatgttcaagttttagttcaactttctcagcggcagaggaaagtgtggtgccccctgcctctcttggacatattaaaaagaggcatgaaatcagcagtcaggttttccatgaatgcttctttaacccttgccacaatagtggaacctgccagcaacttgggcgtggttatgtttgtctctgtccacttggatatacaggcttaaagtgtgaaacagacatcgatgagtgcagcccactgccttgcctcaacaatggagtttgtaaagacctagttggggaattcatttgtgagtgcccatcaggttacacaggtcagcggtgtgaagaaaatataaatgagtgtagctccagtccttgtttaaataaaggaatctgtgttgatggtgtggctggctatcgttgcacatgtgtgaaaggatttgtaggcctgcattgtgaaacagaagtcaatgaatgccagtcaaacccatgcttaaataatgcagtctgtgaagaccaggttgggggattcttgtgcaaatgcccacctggatttttgggtacccgatgtggaaagaacgtcgatgagtgtctcagtcagccatgcaaaaatggagctacctgtaaagacggtgccaatagcttcagatgcctgtgtgcagctggcttcacaggatcacactgtgaattgaacatcaatgaatgtcagtctaatccatgtagaaatcaggccacctgtgtggatgaattaaattcatacagttgtaaatgtcagccaggattttcaggcaaaaggtgtgaaacagaacagtctacaggctttaacctggattttgaagtttctggcatctatggatatgtcatgctagatggcatgctcccatctctccatgctctaacctgtaccttctggatgaaatcctctgacgacatgaactatggaacaccaatctcctatgcagttgataacggcagcgacaataccttgctcctgactgattataacggctgggttctttatgtgaatggcagggaaaagataacaaactgtccctcggtgaatgatggcagatggcatcatattgcaatcacttggacaagtgccaatggcatctggaaagtctatatcgatgggaaattatctgacggtggtgctggcctctctgttggtttgcccatacctggtggtggtgcgttagttctggggcaagagcaagacaaaaaaggagagggattcagcccagctgagtcttttgtgggctccataagccagctcaacctctgggactatgtcctgtctccacagcaggtgaagtcactggctacctcctgcccagaggaactcagtaaaggaaacgtgttagcatggcctgatttcttgtcaggaattgtggggaaagtgaagatcgattctaagagcatattttgttctgattgcccacgcttaggagggtcagtgcctcatctgagaactgcatctgaagatttaaagccaggttccaaagtcaatctgttctgtgatccaggcttccagctggtcgggaaccctgtgcagtactgtctgaatcaaggacagtggacacaaccacttcctcactgtgaacgcattagctgtggggtgccacctcctttggagaatggcttccattcagccgatgacttctatgctggcagcacagtaacctaccagtgcaacaatggctactatctattgggtgactcaaggatgttctgtacagataatgggagctggaacggcgtttcaccatcctgccttgatgtcgatgagtgtgcagttggatcagattgtagtgagcatgcttcttgcctgaacgtagatggatcctacatatgttcatgtgtcccaccgtacacaggagatgggaaaaactgtgcagaacctataaaatgtaaggctccaggaaatccggaaaatggccactcctcaggtgagatttatacagtaggtgccgaagtcacattttcgtgtcaggaaggataccagttgatgggagtaaccaaaatcacatgtttggagtctggagaatggaatcatctaataccatattgtaaagctgtttcatgtggtaaaccggctattccagaaaatggttgcattgaggagttagcatttacttttggcagcaaagtgacatataggtgtaataaaggatatactctggccggtgataaagaatcatcctgtcttgctaacagttcttggagtcattcccctcctgtgtgtgaaccagtgaagtgttctagtccggaaaatataaataatggaaaatatattttgagtgggcttacctacctttctactgcatcatattcatgcgatacaggatacagcttacagggcccttccattattgaatgcacggcttctggcatctgggacagagcgccacctgcctgtcacctcgtcttctgtggagaaccacctgccatcaaagatgctgtcattacggggaataacttcactttcaggaacaccgtcacttacacttgcaaagaaggctatactcttgctggtcttgacaccattgaatgcctggccgacggcaagtggagtagaagtgaccagcagtgcctggctgtctcctgtgatgagccacccattgtggaccacgcctctccagagactgcccatcggctctttggagacattgcattctactactgctctgatggttacagcctagcagacaattcccagcttctctgcaatgcccagggcaagtgggtacccccagaaggtcaagacatgccccgttgtatagctcatttctgtgaaaaacctccatcggtttcctatagcatcttggaatctgtgagcaaagcaaaatttgcagctggctcagttgtgagctttaaatgcatggaaggctttgtactgaacacctcagcaaagattgaatgtatgagaggtgggcagtggaacccttcccccatgtccatccagtgcatccctgtgcggtgtggagagccaccaagcatcatgaatggctatgcaagtggatcaaactacagttttggagccatggtggcttacagctgcaacaaggggttctacatcaaaggggaaaagaagagcacctgcgaagccacagggcagtggagtagtcctataccgacgtgccacccggtatcttgtggtgaaccacctaaggttgagaatggctttctggagcatacaactggcaggatctttgagagtgaagtgaggtatcagtgtaacccgggctataagtcagtcggaagtcctgtatttgtctgccaagccaatcgccactggcacagtgaatcccctctgatgtgtgttcctctcgactgtggaaaacctcccccgatccagaatggcttcatgaaaggagaaaactttgaagtagggtccaaggttcagtttttctgtaatgagggttatgagcttgttggtgacagttcttggacatgtcagaaatctggcaaatggaataagaagtcaaatccaaagtgcatgcctgccaagtgcccagagccgcccctcttggaaaaccagctagtattaaaggagttgaccaccgaggtaggagttgtgacattttcctgtaaagaagggcatgtcctgcaaggcccctctgtcctgaaatgcttgccatcccagcaatggaatgactctttccctgtttgtaagattgttctttgtaccccacctcccctaatttcctttggtgtccccattccttcttctgctcttcattttggaagtactgtcaagtattcttgtgtaggtgggtttttcctaagaggaaattctaccaccctctgccaacctgatggcacctggagctctccactgccagaatgtgttccagtagaatgtccccaacctgaggaaatccccaatggaatcattgatgtgcaaggccttgcctatctcagcacagctctctatacctgcaagccaggctttgaattggtgggaaatactaccaccctttgtggagaaaatggtcactggcttggaggaaaaccaacatgtaaagccattgagtgcctgaaacccaaggagattttgaatggcaaattctcttacacggacctacactatggacagaccgttacctactcttgcaaccgaggctttcggctcgaaggtcccagtgccttgacctgtttagagacaggtgattgggatgtagatgccccatcttgcaatgccatccactgtgattccccacaacccattgaaaatggttttgtagaaggtgcagattacagctatggtgccataatcatctacagttgcttccctgggtttcaggtggctggtcatgccatgcagacctgtgaagagtcaggatggtcaagttccatcccaacatgtatgccaatagactgtggcctccctcctcatatagattttggagactgtactaaactcaaagatgaccagggatattttgagcaagaagacgacatgatggaagttccatatgtgactcctcaccctccttatcatttgggagcagtggctaaaacctgggaaaatacaaaggagtctcctgctacacattcatcaaactttctgtatggtaccatggtttcatacacctgtaatccaggatatgaacttctggggaaccctgtgctgatctgccaggaagatggaacttggaatggcagtgcaccatcctgcatttcaattgaatgtgacttgcctactgctcctgaaaatggctttttgcgttttacagagactagcatgggaagtgctgtgcagtatagctgtaaacctggacacattctagcaggctctgacttaaggctttgtctagagaatagaaagtggagtggtgcctccccacgctgtgaagccatttcatgcaaaaagccaaatccagtcatgaatggatccatcaaaggaagcaactacacatacctgagcacgttgtactatgagtgtgaccccggatatgtgctgaatggcactgagaggagaacatgccaggatgacaaaaactgggatgaggatgagcccatttgcattcctgtggactgcagttcacccccagtctcagccaatggccaggtgagaggagacgagtacacattccaaaaagagattgaatacacttgcaatgaagggttcttgcttgagggagccaggagtcgggtttgtcttgccaatggaagttggagtggagccactcccgactgtgtgcctgtcagatgtgccaccccgccacaactggccaatggggtgacggaaggcctggactatggcttcatgaaggaagtaacattccactgtcacgagggctacatcttgcacggtgctccaaaactcacctgtcagtcagatggcaactgggatgcagagattcctctctgtaaaccagtcaactgtggacctcctgaagatcttgcccatggtttccctaatggtttttcctttattcatgggggccatatacagtatcagtgctttcctggttataagctccatggaaattcatcaagaaggtgcctctccaatggctcctggagtggcagctcaccttcctgcctgccttgcagatgttccacaccagtaattgaatatggaactgtcaatgggacagattttgactgtggaaaggcagcccggattcagtgcttcaaaggcttcaagctcctaggactttctgaaatcacctgtgaagccgatggccagtggagctctgggttcccccactgtgaacacacttcttgtggttctcttccaatgataccaaatgcgttcatcagtgagaccagctcttggaaggaaaatgtgataacttacagctgcaggtctggatatgtcatacaaggcagttcagatctgatttgtacagagaaaggggtatggagccagccttatccagtctgtgagcccttgtcctgtgggtccccaccgtctgtcgccaatgcagtggcaactggagaggcacacacctatgaaagtgaagtgaaactcagatgtctggaaggttatacgatggatacagatacagatacattcacctgtcagaaagatggtcgctggttccctgagagaatctcctgcagtcctaaaaaatgtcctctcccggaaaacataacacatatacttgtacatggggacgatttcagtgtgaataggcaagtttctgtgtcatgtgcagaagggtatacctttgagggagttaacatatcagtatgtcagcttgatggaacctgggagccaccattctccgatgaatcttgcagtccagtttcttgtgggaaacctgaaagtccagaacatggatttgtggttggcagtaaatacacctttgaaagcacaattatttatcagtgtgagcctggctatgaactagaggggaacagggaacgtgtctgccaggagaacagacagtggagtggaggggtggcaatatgcaaagagaccaggtgtgaaactccacttgaatttctcaatgggaaagctgacattgaaaacaggacgactggacccaacgtggtatattcctgcaacagaggctacagtcttgaagggccatctgaggcacactgcacagaaaatggaacctggagccacccagtccctctctgcaaaccaaatccatgccctgttccttttgtgattcccgagaatgctctgctgtctgaaaaggagttttatgttgatcagaatgtgtccatcaaatgtagggaaggttttctgctgcagggccacggcatcattacctgcaaccccgacgagacgtggacacagacaagcgccaaatgtgaaaaaatctcatgtggtccaccagctcacgtagaaaatgcaattgctcgaggcgtacattatcaatatggagacatgatcacctactcatgttacagtggatacatgttggagggtttcctgaggagtgtttgtttagaaaatggaacatggacatcacctcctatttgcagagctgtctgtcgatttccatgtcagaatgggggcatctgccaacgcccaaatgcttgttcctgtccagagggctggatggggcgcctctgtgaagaaccaatctgcattcttccctgtctgaacggaggtcgctgtgtggccccttaccagtgtgactgcccgcctggctggacggggtctcgctgtcatacagctgtttgccagtctccctgcttaaatggtggaaaatgtgtaagaccaaaccgatgtcactgtctttcttcttggacgggacataactgttccaggaaaaggaggactgggttttaaccactgcacgaccatctggctctcccaaaagcaggatcatctctcctcggtagtgcctgggcatcctggaacttatgcaaagaaagtccaacatggtgctgggtcttgtttagtaaacttgttacttggggttactttttttattttgtgatatattttgttattccttgtgacatactttcttacatgtttccatttttaaatatgcctgtattttctatataaaaattatattaaatagatgctgctctaccctcacaaaatgtacatattctgctgtctattgggaaagttcctggtacacatttttattcagttacttaaaatgatttttccattaaagtatattttgctactaaataatattttgctggatagtaccattttatgaggtggccaagggattcatagagaggactcatgatctttcatctgtgccactggcaccacataggagaccccttacaataaaggagacccctttgttctggttccatcttctatacaatatgtgtaataataaaagctttttctttcatttaatcaatcagagagtaactgttggacctggtactctccaccacataattatgtcctggtacttagggtagcctggctaattcttttcacaacatttgaaaagccgtaggcattaacttcatggatgtggagaaacagcataatgaccttggttcacaatgaaagtgctagccttctttctcgtggctggaataagaaatgcaagaactggaagattaacaaagagctgatacccggactggtaatcccacaactcactattttgggaggaaggggaagacagggaaagagaaggaaaaaggcaaatgggaaacattccccagagctgtgctcctggtctcacctgcaaaaaagaattgcagccacctaagagaaagctgagctgcccctcagtcctcactgtcacctacctctatcacaacaattgtaccaagatgaagccttccccagacttcagaaaataaagtcaaaccctagattttgttttaaaataggaaactcagaatcaacttgcctccatcctctgggaaaactgctcccacacaggccttggagtgtgttgtagcactgtggaggaatgcagaaaggatgaaagagatcttgattctcctagtggttctcttcactaccgtaggcatccctcagccattgactcctccttctttctcttgacattcactttcttggccagtcttacatgcttatgagtctactttccaataaatttactcatagtccattaacttccaaaaaaaaaaaaaaa

(19) United States
(12) Patent Application Publication
(10) Pub. No.: US 2015/0086557 A1
(43) Pub. Date: Mar. 26, 2015

(54) NOVEL INTEGRIN alpha9 beta1 LIGAND AND USES THEREOF

(71) Applicant: OSAKA UNIVERSITY, Osaka (JP)

(72) Inventors: Kiyotoshi Sekiguchi, Osaka (JP); Ryoko Sato, Osaka (JP); Sachiko Ezoe, Osaka (JP)

(21) Appl. No.: 14/384,206

(22) PCT Filed: Mar. 14, 2013
(86) PCT No.: PCT/JP2013/057204

§ 371 Date: Sep. 10, 2014

(30) Foreign Application Priority Data
Mar. 15, 2012 (JP) 2012-058795

(51) Int. Cl. C07K 7/06; C12N 5/071; G01N 33/50; C07K 16/18; C07K 7/08

(52) U.S. Cl. CPC C07K 7/06; C07K 16/18; C07K 7/08; G01N 33/502; C12N 5/0602; G01N 2500/02
USPC 424/139.1; 530/328; 536/23.5; 530/329; 530/327; 514/21.6; 435/320.1; 435/29; 435/377

(57) ABSTRACT

Provided is a novel ligand for integrin α9β1 consisting of a peptide having the following amino acid sequence:
(A) EDDMMEVPY (SEQ ID NO: 1) or
(B) an amino acid sequence the same as the amino acid sequence represented by SEQ ID NO: 1 except for having deletion, substitution or addition of 1 to 3 amino acids. The novel ligand for integrin α9β1 has a higher binding affinity than those of tenascin-C and osteodontin, which are known ligands for integrin α9β1.

### TECHNICAL FIELD

The present invention relates to a novel ligand for integrin α9β1 and use thereof.

### BACKGROUND ART

The extracellular matrix (hereinafter referred to as “ECM”) is a structure surrounding cells and plays an essential role as cells' immediate microenvironment in maintaining cellular survival and controlling cellular proliferation and differentiation. Integrins are major ECM receptors on the cell surface and have a number of isoforms with different subunit compositions. In humans, 24 kinds of integrins exist and 18 kinds of them function as ECM receptors. Most of these integrins have a β1 chain and, depending on the type of α chain, are roughly classified into a “collagen-binding subgroup (α1β1, α2β1, etc ) , ” a “laminin-binding subgroup (α3β1, α6β1, etc.),” and an “Arg—Gly—Asp (RGD) sequence-binding subgroup (α5β1, α8β1, etc.)” However, as for some integrins such as α9β1, their true ligands still remain unidentified.

Integrin α9β1 is expressed in epithelial cells, smooth muscle cells, vascular endothelial cells, neutrophils, etc., and reportedly involved in angiogenesis, lymphangiogenesis, wound healing and autoimmune arthritis. In addition, integrin α9β1 is expressed in corneal stem cells and hematopoietic stem cells, and may be involved in maintaining she functions of these tissue stem cells. The so far reported ligands for integrin α9β1 include tenascin-C, osteopontin and cellular fibronectin (these are ECM proteins); VCAM-1 (vascular cell adhesion molecule 1) and ADAM protease (these are membrane proteins); and VEGF (vascular endothelial growth factor) and NGF (nerve growth factor) (these are growth factors) (see Non Patent Literature 1 to 7). However, the binding affinities of these proteins for integrin α9β1 are markedly lower than those between laminin-binding or RGD-binding integrins and their respective ligands, and the measurement is often based on cell adhesion activity, not direct integrin α9β1-binding activity. In addition, regarding tenascin-C and osteopontin as typical ECM ligands for integrin α9β1, the ligand activities can be detected only in their specific fragments, not in their full-length proteins. Therefore, it still remains uncertain whether they function as physiological ligands. For the reasons above, it has been thought that true ECM ligands for integrin α9β1 are other than those proteins.

### CITATION LIST

### Non Patent Literature

Non Patent Literature 1:

Yokosaki, Y., Palmer, E. L., Prieto, A. L,, Crossin, K. L., Bourdon, M. A., Pytela, R., and Sheppard, D. (1994) J. Biol. Chem, 269, 26691-26696

Non Patent Literature 2:

Smith, L. L., Cheung, H. K., Ling, L. E., Chen, J., Sheppard, D., Pytela, R., and Giachelli, C. M. (1996) J. Biol. Chem. 271, 28485-28491

Non Patent Literature 3:

Liao, Y. F., Gotwals, P. J., Koteliansky, V. E., Sheppard, D., and Van De Water, L. (2002) J. Biol. Chem. 277, 14467-14474

Non Patent Literature 4:

Eto, K., Huet. C., Tarui, T., Kupriyanov, S., Liu, H. Z., Puzon-McLaughlin, W., Zhang, X. P., Sheppard, D., Engvall, E., and Takada, Y. (2002) J. Biol. Chem. 277, 17804-17810

Non Patent Literature 5:

Taooka, Y., Chen, J., Yednock, T., and Sheppard, D. (1999) J. Cell Biol. 145, 413-420

Non Patent Literature 6:

Vlahakis, N. E., Young, B. A., Atakilit, A., and Sheppard, D. (2005) J. Biol. Chem. 280, 4544-4552

Non Patent Literature 7:

Staniszewska, I., Sariyer, I. K., Lecht, S., Brown, M. C., Walsh, E. M., Tuszynski, G. P., Safak, M., Lazarovici, P., and Marcinkiewicz, C. (2008) J. Cell Sci. 121, 504-513

### SUMMARY OF INVENTION

### Technical Problem

An object of the present invention is to provide a novel ligand for integrin α9β1 which has a higher binding affinity than those of tenascin-C and osteopontin, which are known ligands for integrin α9β1; and usage thereof.

### Solution to Problem

The present invention includes the following to achieve the above-mentioned object.

A ligand for integrin α9β1, consisting of a peptide having the following amino acid sequence:

(A) EDDMMEVPY (SEQ ID NO: 1) or

(B) an amino acid sequence the same as the amino acid sequence represented by SEQ ID NO: 1 except for having deletion, substitution or addition of 1 to 3 amino acids.

[2] The ligand for integrin α9β1 according to the above [1], wherein the ligand is SVEP1 or an active fragment thereof.

[3] The ligand for integrin α9β1 according to the above [1] or [2], wherein the ligand has 2500 amino acid residues or less.

[4] A polynucleotide encoding a peptide constituting the ligand for integrin α9β1 according to any one of the above [1] to [3].

[5] An expression vector containing the polynucleotide according to the above [4].

[6] An antibody against the ligand for integrin α9β1 according to any one of the above [1] to [3].

[7] A method for screening for substances capable of inhibiting interaction of SVEP1 with integrin α9β1, the method using the ligand for integrin α9β1 according to any one of the above [1] to [3].

[8] A method for culturing integrin α9β1-expressing cells, the method using the ligand for integrin α9β1 according to any one of the above [1] to [3] as a substrate.

[9] A method for culturing tissue stem cells with maintenance of their undifferentiated state and inhibition of their proliferation, the method using the ligand for integrin α9β1 according to any one of the above [1] to [3] as a substrate.

[10] A method for separating integrin α9β1-expressing cells, the method allowing selective separation of cells capable of binding to the ligand for integrin α9β1 according to any one of the above [1] to [3].

[11] A drug delivery system for delivering a drug to integrin α9β1-expressing cells, the system comprising the ligand for integrin α9β1 according to any one of the above [1] to [3], and the drug.

[12] A method for inducing tissue stem cells in a resting phase or an antiproliferative state into a proliferative state, the method comprising bringing the stem cells into contact with the ligand for integrin α9β1 according to any one of the above [1] to [3].

### Advantageous Effects of Invention

According to the present invention, a novel ligand for integrin α9β1 can be provided. The novel ligand for integrin α9β1 has a higher binding affinity than those of known ligands for integrin α9β1. The novel ligand for integrin α9β1 can be preferably used for a method for screening for substances capable of inhibiting the interaction of SVEP1 with integrin α9β1; a method for culturing integrin α9β1-expressing cells; a method for culturing hematopoietic stem cells with maintenance of their undifferentiated state and inhibition of their proliferation; a method for delivering a drug to integrin α9β1-expressing cells; a method for inducing stem cells in the resting phase or an antiproliferative state into a proliferative state; and other purposes.

### BRIEF DESCRIPTION OF DRAWINGS

FIG. 1 shows the domain structure of mouse SVEP1 (referred to as “polydom” in the Drawings and the Brief Description of Drawings).

FIG. 2 shows the results of 6% non-reducing or reducing SDS-PAGE for a recombinant polydom having the signal sequence of the mouse immunoglobulin κ chain.

FIG. 3 shows the results of immunoblotting for a recombinant polydom having an N-terminal FLAG tag and a C-terminal His tag. (A) shows the results of immunoblotting using an anti-His antibody, and (B) shows the results of immunoblotting using an anti-FLAG antibody.

FIG. 4 shows the results of the examination of adhesion of RD cells to polydom.

FIG. 5 shows the results of microscopic observation of adhesion of RD cells onto different substrates.

FIG. 6 shows the results of the examination on integrin dependence of adhesion of RD cells onto polydom.

FIG. 7 shows the results of the examination of binding between recombinant integrin α9β1 and polydom.

FIG. 8 shows the results of the examination of binding between integrin α9β1 and polydom or other known ligands for integrin α9β1.

FIG. 9 shows N-terminal deletion mutants of polydom prepared for identification of the integrin α9β1-binding site in polydom.

FIG. 10 shows the results of the evaluation of the integrin α9β1-binding activities of N-terminal deletion mutants of polydom.

FIG. 11 shows the alignment of the amino acid sequences of the 20th, 21st and 22nd CCP domains by ClustalW.

FIG. 12 shows the results of the examination of the integrin α9β1-binding site in the 21st CCP domain (hereinafter referred to as “CCP21”).

FIG. 13 shows the results of the examination of the integrin α9β1-binding site in the extra 37-amino acid segment (D2628-S2664) of CCP21.

FIG. 14 shows the results of the examination using alanine scanning mutants on the contribution of the amino acid residues in the extra 12-amino acid segment (D2634-L2645) of CCP21 to the binding to integrin α9β1.

FIG. 15 shows the results of the integrin α9β1-pol-C binding inhibition assay using synthetic peptides consisting of the EDDMMEVPY sequence or a part of the sequence.

FIG. 16 shows the localization of polydom and integrin α9 as revealed by immunofluorescence staining of mouse embryonic tissues.

FIG. 17 shows the results of the in situ integrin binding assay using cryosections of mouse embryos.

FIG. 18 shows the results of the examination of proliferation of hematopoietic stem cells on polydom or fibronectin.

FIG. 19 shows the results of the examination of differentiation of hematopoietic stem cells on polydom or fibronectin.

FIG. 20 shows the results of the examination of the effects of the EDDMMEVPY peptide on proliferation of hematopoietic stem cells on polydom.

FIG. 21 shows the results of fractionation of cardiac cells based on the fluorescence of green fluorescent protein (GFP) by cell sorter FACSAria. The cardiac cells were prepared 6 weeks after a fusion protein of GFP and histone H2B (H2B-GFP) was expressed in the heart of a mouse for 2 weeks starting 1 week before birth.

FIG. 22 shows the results of integrin α9 expression in the GFP-positive or GFP-non-positive cells fractionated as shown in FIG. 21, as analyzed by cell sorter FACSAria.

FIG. 23 shows the localization of GFP label-retaining cells and integrin α9 expressing cells in the cryosections of a mouse heart containing H2B-GFP-labeled cardiac stem cells, as revealed by double fluorescence staining.

FIG. 24 shows the localization of GFP label-retaining cells and polydom expressing regions in the cryosections of a mouse heart containing H2B-GFP-labeled cardiac stem cells.

FIG. 25 shows the results of the examination of the cell adhesion activities of polydom and fibronectin for H2B-GFP-labeled cardiac stem cells (GFP label-retaining cells).

FIG. 26 shows the results of the examination of the colony forming ability of H2B-GFP-labeled cardiac stem cells (GFP label-retaining cells) on polydom or fibronectin. (A) shows the colony forming activity, and (B) shows typical colony morphologies.

### DESCRIPTION OF EMBODIMENTS

The present inventors previously established a bioinformatics-based protocol for screening for ECM proteins and identified 16 novel ECM proteins from among more than 65000 varieties of mouse cDNAs available in the Riken FANTOM cDNA collection (Manabe, R., Tsutsui, K., Yamada, T., Kimura, M., Nakano, I., Shimono, C., Sanzen, N., Furutani, Y., Fukuda, T., Oguri, Y., Shimamoto, K., Kiyozumi, D., Sato, Y., Sado, Y., Senoo, H., Yamashina. S., Fukuda, S., Kawai, J., Sugiura, N., Kimata, K., Hayashizaki, Y., and Sekiguchi, K. (2008) Proc. Natl. Acad. Sci. U. S. A, 105, 12849-12854). The present inventors then extended the screening using this protocol to mouse and human genome databases, and focusing on transcripts encoding proteins of 1500 or more amino acid residues, identified more ECM proteins. One of the obtained candidates was SVEP1 (also called polydom). As a result of intensive research, the present inventors found that SVEP1 is a ligand for integrin α9β1. Subsequently, the present inventors found that SVEP1 is cleaved into an N-terminal fragment and a C-terminal fragment after secretion and that the C-terminal fragment has a ligand activity for integrin α9β1. Next, the present inventors found that the integrin α9β1 recognition sequence is present in the 21st complement control protein (CCP) domain from the N-terminus (CCP21) of mouse SVEP1, which contains 34 CCPs. The present inventors further found that an extra segment is present in CCP21, not in the other CCPs of mouse SVEP1 and that the integrin α9β1 recognition sequence is the amino acid sequence EDDMMEVPY (SEQ ID NO: 1) in the extra segment.

### Ligand for Integrin α9β1

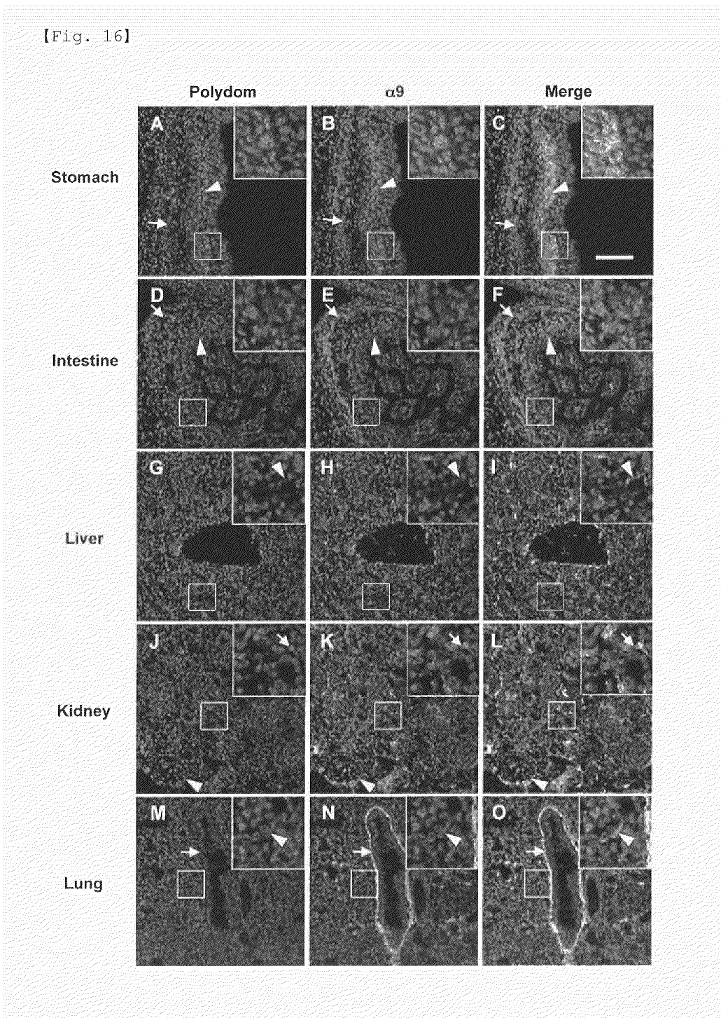

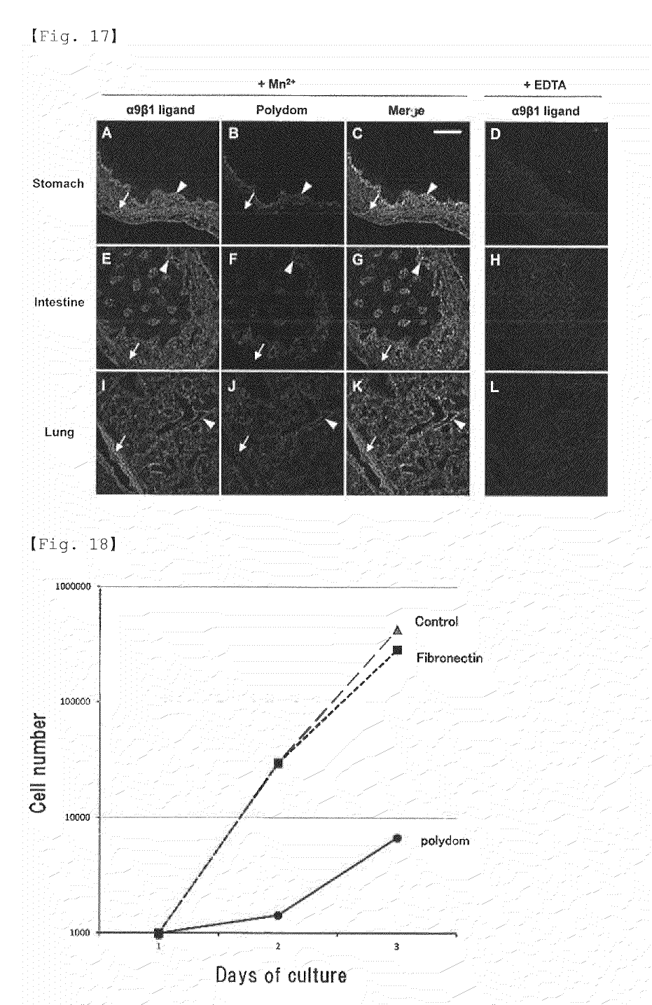

The present invention provides a ligand for integrin α9β1 consisting of a peptide having the amino acid sequence EDDMMEVPY (SEQ ID NO: 1). The present invention also provides a ligand for integrin α9β1 consisting of a peptide having an amino acid sequence the same as the amino acid sequence represented by SEQ ID NO: 1 except for having deletion, substitution or addition of 1 to 3 amino acids (hereinafter also referred to as “mutant of the sequence of SEQ ID NO: 1”). The present inventors examined the integrin α9β1 recognition sequence using deletion mutants, and as a result, found that deletion of E at residue 1 and deletion of D at residue 2 in the amino acid sequence represented by SEQ ID NO: 1 did not compromise the integrin α9β1-binding activity, and that further deletion of Y at residue 9 reduced the integrin α9β1-binding activity but did not abrogate it (see FIG. 16). The present inventors also used alanine scanning mutants to examine the integrin α9β1 recognition sequence. The examination results showed that alanine substitution of M at residue 4 hardly altered the integrin α9β1-binding activity and that alanine substitution of E at residue 1, P at residue 8, or the like had only a small contribution to reduction in the binding activity (see FIG. 14). Therefore, peptides or proteins having a mutant of the sequence of SEQ ID NO: 1 have a ligand activity for integrin α9β1.

The ligand of the present invention for integrin α9β1 (hereinafter referred to as “ligand of the present invention”) is not limited as long as the ligand consists of a peptide having the amino acid sequence represented by SEQ ID NO: 1 or a mutant of the sequence of SEQ ID NO: 1. That is, the peptide constituting the ligand of the present invention may consist of the amino acid sequence represented by SEQ ID NO: 1 or a mutant of the sequence of SEQ ID NO: 1, or comprise the amino acid sequence represented by SEQ ID NO: 1 or a mutant of the sequence of SEQ ID NO: 1, and an additional amino acid sequence other than both sequences. The additional amino acid sequence other than the amino acid sequence represented by SEQ ID NO: 1 and a mutant of the sequence of SEQ ID NO: 1 is not particularly limited and the number of its amino acid residues is also not limited. The “peptide” as used herein means a molecule of two or more amino acids bound together via a peptide bond(s) and the number of the amino acids bound together is not limited. That is, the “peptide” as used herein encompasses polypeptides.

In the amino acid sequence represented by SEQ ID NO: 1, E at residue 1 is considered to be substitutable by any amino acid. D at residue 2 is considered to be substitutable by any amino acid, preferably an acidic amino acid. D at residue 3 is considered to be substitutable by E. M at residue 4 is considered to be substitutable by any amino acid. M at residue 5 is considered to be substitutable by another hydrophobic amino acid. E at residue 6 is considered to be substitutable by D. V at residue 7 is considered to be substitutable by any amino acid, preferably a hydrophobic amino acid. P at residue 8 is considered to be substitutable by any amino acid, but preferably remains as it is. Y at residue 9 is considered to be substitutable by any amino acid, but preferably remains as it is.

The ligand of the present invention is preferably full-length SVEP1 or an active fragment thereof. SVEP1 is a secretory protein present mainly in vertebrate ECM and has a number of domains. As shown in FIG. 1, mouse SVEP1 (full-length) has a signal sequence, a von Willebrand factor A domain (vWFA), an Eph-like domain, CCP domains, HYR domains, STT2R domains, EGF domains and a PTX domain. The domain structures of SVEP 1 proteins of other mammals are the same as that shown in FIG. 1. The amino acid sequence represented by SEQ ID NO: 1 is well conserved in mammals. Therefore, the ligand of the present invention is preferably a mammalian SVEP1 or an active fragment thereof, and more preferably human SVEP1 or an active fragment thereof.

The accession numbers for the SVEP1 amino acid sequences and the nucleotide sequences of the SVEP1-encoding genes in typical mammals are shown in Table 1. These amino acid and nucleotide sequence data can be obtained from known databases (GenBank etc.). Each of the mammalian SVEP1 proteins shown in Table 1 has the amino acid sequence represented by SEQ ID NO: 1, and the amino acid sequence represented by SEQ ID NO: 1 is present in a CCP domain containing an extra segment, specifically present in the extra segment. The SVEP1 as the ligand of the present invention is not limited to these examples, and SVEP1 proteins of other animals are also considered suitable as the ligand for integrin α9β1. The amino acid and nucleotide sequence data of SVEP1 proteins of other animals can be obtained from known databases (GenBank etc.). The amino acid sequence of mouse SVEP1 is shown in SEQ ID NO: 2, the nucleotide sequence of mouse SVEP1 is shown in SEQ ID NO: 3, the amino acid sequence of human SVEP1 is shown in SEQ ID NO: 4, and the nucleotide sequence of human SVEP1 is shown in SEQ ID NO: 5.

TABLE 1
Sequence data of SVEP1 (accession numbers)

| | Amino acid sequence | Nucleotide sequence |
|---|---|---|
| Mouse | NP_073725 | NM_022814 |
| Human | NP_699197 | NM_153366 |
| Chimpanzee | XP_520182 | XM_520182 |
| Dog | XP_532030 | XM_532030 |
| Cattle | XP_002689967 | XM_002689921 |
| Horse | XP_001916184 | XM_001916149 |
| Pig | XP_003122123 | XM_003122075 |
| Rat | XP_001065678 | XM_001065678 |
| Rabbit | XP_002708118 | XM_002708072 |

The active fragment of SVEP1 as the ligand for integrin α9β1 may be any fragment that consists of a part of SVEP1 and has a ligand activity for integrin α9β1 (binding activity for integrin α9β1). Preferable examples of the active fragment of SVEP1 include a SVEP1 fragment having the amino acid sequence represented by SEQ ID NO: 1 or a mutant of the sequence of SEQ ID NO: 1. The specific examples include a mammalian SVEP1 fragment corresponding to the C-terminal fragment of mouse SVEP1 represented as “pol-C” in FIG. 1; a mammalian SVEP1 fragment having a CCP domain containing an extra segment; a mammalian SVEP1 fragment having the extra segment; a SVEP1 fragment having the amino acid sequence represented by SEQ ID NO: 1; and a SVEP1 fragment having a mutant of the sequence of SEQ ID NO: 1.

The number of amino acid residues of the peptide constituting the ligand of the present invention is not particularly limited, but preferably not more than about 2500 residues. The lower limit is 6 residues, which is the number when 3 amino acids are deleted from the amino acid sequence represented by SEQ ID NO: 1. Therefore, the number of amino acid residues of the peptide constituting the ligand of the present invention is preferably 6 to about 2500 residues, more preferably 6 to about 2000 residues, further preferably 6 to about 1500 residues, further preferably 6 to about 1000 residues, further preferably 6 to about 500 residues, further preferably 6 to about 200 residues, further preferably 6 to about 100 residues, further preferably 6 to about 50 residues, further preferably 6 to about 20 residues, and most preferably 9 residues.

The ligand of the present invention can be prepared, for example, by using known genetic engineering techniques, specifically by constructing a recombinant expression vector having an expressible insert of a gene encoding SVEP1 or an active fragment thereof, transfecting the vector into appropriate host cells for expression of a recombinant protein, and purifying the recombinant protein. Alternatively, for example, in vitro coupled transcription-translation system can also be used for the preparation of the ligand of the present invention. Also, according to a known ordinary peptide synthesis protocol, the ligand of the present invention can be prepared by a solid phase synthesis method (the Fmoc method, the Boc method) or a liquid phase synthesis method.

The C-terminus of the peptide may be a carboxyl group (—COOH), a carboxylate (—COO⁻), an amide (—CONH₂) or an ester (—COOR). Examples of the R moiety in the ester include C₁₋₆ alkyl groups such as methyl, ethyl, n-propyl, isopropyl and n-butyl; C₃₋₈ cycloalkyl groups such as cyclopentyl and cyclohexyl; C₈₋₁₂ aryl groups such as phenyl and α-naphthyl; C₇₋₁₄ aralkyl groups such as phenyl-C₁₋₂ alkyl groups including benzyl and phenethyl, and α-naphthyl-C₁₋₂ alkyl groups including α-naphthylmethyl; and a pivaloyloxymethyl group, which is widely used as an ester for oral use. When the peptide has a carboxyl group or a carboxylate in a site other than the C-terminus, these groups may be amidated or esterified.

The amino acids constituting the peptide may have a substituent in the side chain. The substituent is not particularly limited and the examples include a fluorine atom, a chlorine atom, a cyano group, a hydroxyl group, a nitro group, an alkyl group, a cycloalkyl group, an alkoxy group and an amino group. Also, in the peptide, the amino group of the N-terminal amino acid residue may be protected by a protecting group (for example, C₁₋₆ acyl groups including a formyl group and a C₂₋₆ alkanoyl group such as acetyl, etc.); a glutamyl group resulting from in vivo N-terminal cleavage of the peptide may be pyroglutamated; and a side chain substituent(s) (for example, —OH, —SH, an amino group, an imidazole group, an indole group, a guanidino group, etc.) of the intramolecular amino acid(s) may be protected by an appropriate protecting group(s) (for example, C₁₋₆ acyl groups including a formyl group and a C₂₋₆ alkanoyl group such as acetyl, etc.).

The peptide may be in the form of a pharmaceutically acceptable salt. Examples of the salt include a salt formed with an acid, such as hydrochloric acid, sulfuric acid, phosphoric acid, lactic acid, tartaric acid, maleic acid, fumaric acid, oxalic acid, malic acid, citric acid, oleic acid and palmitic acid; a salt formed with a hydroxide or a carbonate of an alkali or alkaline earth metal such as sodium, potassium and calcium, and a salt formed with a hydroxide or a carbonate of aluminum; and a salt formed with triethylamine, benzylamine, diethanolamine, tert-butylamine, dicyclohexylamine, arginine or the like.

### Polynucleotide

The polynucleotide of the present invention may be any polynucleotide that encodes the peptide constituting the ligand of the present invention. The polynucleotide can be present in the form of RNA (for example, mRNA) or DNA (for example, cDNA or genomic DNA). The polynucleotide may be a double or single strand. The double strand may be a double-stranded DNA, a double-stranded RNA or a DNA-RNA hybrid. The single strand may be a coding strand (sense strand) or a non-coding strand (antisense strand). The polynucleotide of the present invention may be fused with a polynucleotide encoding a tag for labeling (a tag sequence or a marker sequence) at the 5′- or 3′-terminus. The polynucleotide of the present invention may further contain an untranslated region (UTR) sequence, a vector sequence (including an expression vector sequence), etc.

The polynucleotide of the present invention can be obtained by a known DNA synthesis method, PCR, etc. To be more specific, in one example, based on the amino acid sequence of the peptide constituting the ligand of the present invention, the nucleotide sequence is designed by appropriate selection of the codon of each amino acid, and the designed nucleotide sequence is chemically synthesized on a commercial DNA synthesizer to give the polynucleotide of the present invention. In another example, primers are designed for amplifying a region which encodes the peptide constituting the ligand of the present invention in the nucleotide sequence of the SVEP1-encoding gene (see SEQ ID NOS: 3 and 5, and the accession numbers in Table 1), and using these primers and a template such as genomic DNA and cDNA of the same animal origin, PGR is performed to give a DNA fragment containing the polynucleotide of the present invention in a large amount.

### Expression Vector

The present invention provides an expression vector used for the production of the ligand of the present invention. The expression vector of the present invention is not particularly limited as long as it contains a polynucleotide encoding the peptide constituting the ligand of the present invention, but preferred are plasmid vectors carrying a recognition sequence for RNA polymerase (pSP64, pBluescript, etc.). The method for preparing the expression vector is not particularly limited, and the expression vector may be prepared with the use of a plasmid, a phage, a cosmid or the like. The kind of the vector is not particularly limited and any appropriate vector that can be expressed in host cells can be selected. For example, depending on the kind of the host cell, an appropriate promoter sequence to ensure the expression of the polynucleotide of the present invention is selected, and this promoter sequence and the polynucleotide of the present invention are inserted into a plasmid etc. to prepare the desired expression vector. After a host transformed with the expression vector of the present invention is cultured, cultivated or bred, the peptide constituting the ligand of the present invention can be collected and purified from the culture products etc. by conventional methods (for example, filtration, centrifugation, cell disruption, gel filtration chromatography, ion exchange chromatography, etc.).

The expression vector preferably contains at least one selection marker. Examples of the marker include a dihydrofolate reductase gene and a neomycin resistance gene for eukaryote cell culture; and a tetracycline resistance gene and an ampicillin resistance gene for culture of Escherichia coli (E. coli) and other bacteria. Such a selection marker is useful for checking whether the polynucleotide of the present invention has been successfully transfected into host cells and is reliably expressed therein. Also, the peptide constituting the ligand of the present invention may be expressed as a fusion peptide. For example, green fluorescent protein (GFP) derived from Aequorea coerulescens is used as a marker and the peptide constituting the ligand of the present invention is expressed as a GFP fusion peptide.

The host is not particularly limited and various known cells can be preferably used. Specific examples of the host include bacteria such as E. coli, yeasts (budding yeast Saccharomyces cerevisiae and fission yeast Schizosaccharomyces pombe), nematodes (Caenorhabditis elegans), Xenopus laevis oocytes and animal cells (for example, CHO cells, COS cells and Bowes melanoma cells). The method for transfecting the above-mentioned expression vector into host cells, i.e. the transformation method, is also not particularly limited and known methods such as electroporation, the calcium phosphate method, the liposome method and the DEAE dextran method can be preferably used. The thus-obtained transformant is also included in the present invention.

### Antibody

The present invention provides an antibody against the ligand of the present invention. The antibody against the ligand of the present invention may be a polyclonal or monoclonal antibody. The antibody may also be a complete antibody molecule or an antibody fragment capable of specifically binding to an antigen of interest (for example, Fab, F (ab′)₂, Fab′, Fv, scFv, etc.). The polyclonal antibody can be obtained in the following manner, for example. An antigen (the peptide constituting the ligand of the present invention) is dissolved in PBS and if needed further mixed with an appropriate amount of a usual adjuvant (for example. Freund's complete adjuvant) to prepare an immunogen, and a mammal (a mouse, a rat, a rabbit, a goat, a horse, etc) is immunized with the immunogen. The method for the immunization is not particularly limited, but preferred is subcutaneous or intraperitoneal injection given once or repeated several times at appropriate intervals, for example. Then, blood collection from the immunized animal, serum separation and purification of polyclonal antibody fractions are performed in a usual manner. The monoclonal antibody can be obtained by fusing immune cells (for example, splenocytes) obtained from the above-mentioned immunized mammal with myeloma cells to produce a hybridoma, culturing the hybridoma and collecting an antibody from the culture. A recombinant monoclonal antibody can also be produced by using recombinant techniques, specifically by cloning an antibody gene from the hybridoma, inserting the gene into a suitable vector and transfecting the vector into host cells. A phage display method can also be used for production of the monoclonal antibody.

### Screening Method

The present invention provides a method for screening for substances capable of inhibiting the interaction of SVEP1 with integrin α9β1. The screening method of the present invention is not limited as long as the method uses the ligand of the present invention. The test substance to be screened is not particularly limited and the examples include nucleic acids, peptides, proteins, non-peptidic compounds, synthetic compounds, fermentation products, cell extracts, cell culture supernatants, plant extracts, mammalian tissue extracts and plasma.

The details of the screening method are not particularly limited. For example, in the case where the binding level of the ligand of the present invention and integrin α9β1-expressing cells brought into contact with a test substance is reduced as compared with that of the ligand of the present invention and integrin α9β1-expressing cells not brought into contact with the test substance, the test, substance is selected as a substance capable of inhibiting the interaction of SVEP1 with integrin α9β1. The degree of reduction of the binding level by a test substance to be selected is not particularly limited, and for example, the binding level is reduced to preferably 50% or less and more preferably 25% or less relative to that of the cells not brought into contact with the test substance.

The substance selected by the screening method of the present invention can inhibit the interaction of SVEP1 with integrin α9β1, and thus is useful as, for example, a proliferation inducer for stem cells in the resting phase or an antiproliferative state, a therapeutic medicine of autoimmune arthritis, etc.

### Culture Method

The present invention provides a method for culturing integrin α9β1-expressing cells. The culture method of the present invention is not limited as long as the method uses the ligand of the present invention as a substrate. The ligand of the present invention is discovered based on the ECM protein SVEP1 and thus is very useful as a substrate for culture of integrin α9β1-expressing cells. In an example of the culture method of the present invention, integrin α9β1-expressing cells are cultured on a culture plate coated with the ligand of the present invention. Examples of the integrin α9β1-expressing cells include tissue stem cells such as hematopoietic stem cells, corneal stem cells and cardiac stem cells, epithelial cells, smooth muscle cells, vascular endothelial cells, hepatocytes and neutrophils. The medium to be used is not particularly limited and any appropriate medium for the cells to be cultured may be selected. As needed, growth factors such as serum can be added depending on the purpose.

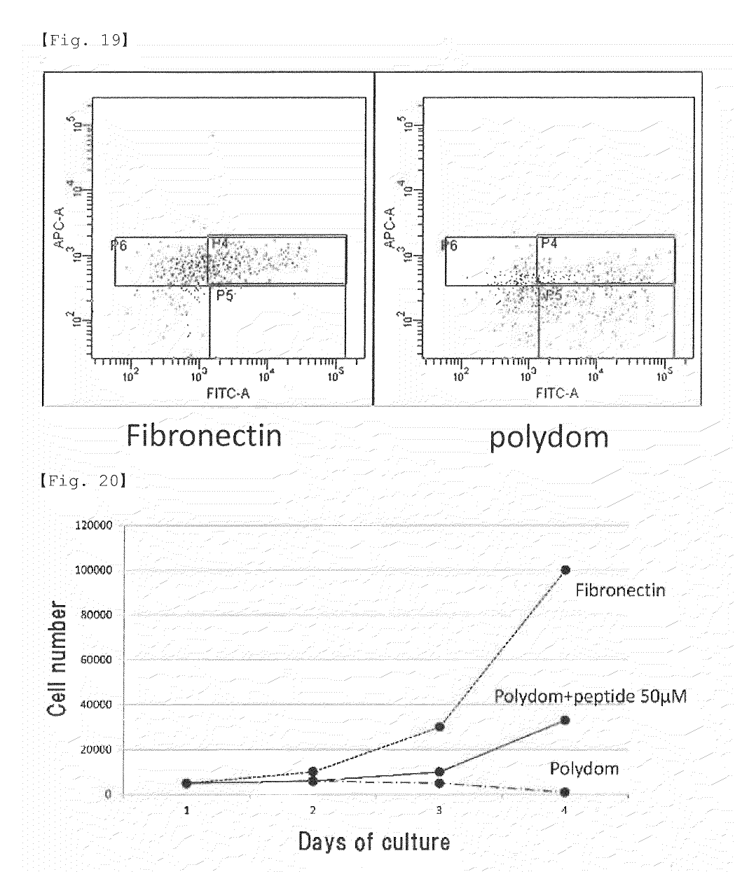

The present invention also provides a method for culturing tissue stem cells with maintenance of their undifferentiated state and inhibition of their proliferation. The method of the present invention for culturing tissue stem cells is not limited as long as the method uses the ligand of the present invention as a substrate. The present inventors found that hematopoietic stem cells cultured on a culture substrate coated with the integrin α9β1 ligand SVEP1 were remarkably inhibited from proliferating and maintained in their undifferentiated state in comparison with those cultured on a culture substrate coated with PBS (control) or fibronectin (see FIGS. 18 and 19). Therefore, the ligand of the present invention is very useful as a substrate for culturing tissue stem cells with maintenance of their undifferentiated state and inhibition of their proliferation. The medium used for the method for culturing tissue stem cells with maintenance of their undifferentiated state and inhibition of their proliferation is not particularly limited as long as the medium is suitable for culture of tissue stem cells. However, it is preferable that growth factor-free media such as serum-free media are used.

### Separation Method

The present invention provides a method for separating integrin α9β1-expressing cells. The separation method of the present invention is not limited as long as the method allows selective separation of cells capable of binding to the ligand of the present invention. Since the ligand of the present invention is capable of binding to integrin α9β1-expressing cells, selective separation of cells bound to the ligand of the present invention enables isolation and concentration of integrin α9β1-expressing cells. The means for selective separation of the cells bound to the ligand of the present invention is not particularly limited and known means for cell separation can be preferably used. Examples of the known means include magnetic beads onto which the ligand of the present invention is immobilized, resins onto which the ligand of the present invention is immobilized, and a cell sorter. Examples of the integrin α9β1-expressing cells to be separated include tissue stem cells such as hematopoietic stem cells, corneal stem cells and cardiac stem cells, smooth muscle cells, vascular endothelial cells, hepatocytes and neutrophils.

### Drug Delivery System

The present invention provides a drug delivery system which comprises the ligand of the present invention and a drug and is intended for delivering the drug to integrin α9β1-expressing cells. Since the ligand of the present invention has an integrin α9β1-binding activity, it can be preferably used in a drug delivery system for delivering a drug to integrin α9β1-expressing cells. Examples of the integrin α9β1-expressing cells include tissue stem cells such as hematopoietic stem cells, corneal stem, cells and cardiac stem cells, epithelial cells, smooth muscle cells, vascular endothelial cells, hepatocytes and neutrophils.

The drug delivery system of the present invention may be in the form of a composition in which the ligand of the present invention and the drug are mixed but nor integrated with each other, but preferably in the form in which the ligand of the present invention and the drug are integrated with each other via soma interaction, The means for such integration is not particularly limited, and the drug delivery system may be in the form of, for example, a complex of the ligand of the present invention and a liposome encapsulating the drug, or a complex of the ligand of the present invention and a nano material carrying the drug. Another possible form is the one in which the ligand of the present invention and the drug are directly bound to each other. In the case where the drug is a protein, a fusion protein of the drug and the ligand of the present invention is suitable.

The drug used in the drug delivery system of the present invention is not particularly limited as long as it is preferable to deliver the drug to target integrin α9β1-expressing cells. Examples of the drug include drugs for disease treatment and drugs for visualization of integrin α9β1-expressing cells (a fluorescent substance, a radioactive substance, a chemiluminescent substance, a magnetic substance, etc.).

### Proliferation Inducing Method

The present invention provides a method for inducing stem cells in the resting phase or an antiproliferative state into a proliferative state by bringing the stem cells into contact with the ligand of the present invention. The present inventors found that although hematopoietic stem cells cultured on a culture substrate coated with the integrin α9β1 ligand SVEP1 was remarkably inhibited from proliferating, the antiproliferative state of hematopoietic stem cells on a SVEP1-coated culture substrate was canceled by treatment with a peptide consisting of the amino acid sequence represented by SEQ ID NO: 1, which is an integrin α9β1 ligand (see FIG. 20), Therefore, the ligand of the present invention can be preferably used for the applications requiring cancellation of the undifferentiated state of hematopoietic stem cells and induction of the cells into a proliferative state.

The application of the proliferation inducing method of the present invention to leukemia patients holds promise for improving the effectiveness of leukemia treatment. That is, the application of the proliferation inducing method enables leukemic stem cells in the resting phase or an antiproliferative state present in the bone marrow of leukemia patients to be induced into an antiproliferative state and thereby migrated into a blood flow, and subsequent administration of a medicine having specific effect on leukemia cells (tyrosine kinase inhibitors etc.) can potentially result in efficient killing of the leukemic stem cells.

### Other Applications

The ligand of the present invention is useful for other applications, for example, sis a reagent for integrin α9β1 activation signaling studies, a reagent for collection of integrin α9β1-expressing cells, etc.

### EXAMPLES

Hereinafter, the present invention will be illustrated in detail by examples, but is not limited thereto.

### Example 1

### Identification of Novel Ligand for Integrin α9β1

### Experimental Procedures

(1) cDNA Cloning and Construction of Expression Vectors

A DNA segment encoding a FLAG tag and a DMA segment encoding a 6×His tag were PCR-amplified. These amplified segments were separately inserted into the NotI/ApaI site of the pcDNA3.1 (+) vector or the pSecTag2B vector (these vectors are available from Invitrogen), yielding pcDNA-FLAG, pSec-FLAG and pSec-His. For construction of expression vectors for N-terminal FLAG-tagged proteins, the DNA segment encoding the FLAG sequence was inserted into the HindIII/BamHI site of the pSec-His vector to yield pSec-NFLAG-His. A cDNA encoding mouse SVEP1 (hereinafter referred to as “polydom” in Examples) was obtained by RT-PCR using RNA extracted from mouse embryos at embryonic day 7 (Clontech) and subcloned into pBluescript KS(+) (Stratagene). After verification of the nucleotide sequence, an error-free cDNA fragment was inserted into pcDNA-FLAG, pSec-FLAG, pSec-His or pSec-NFLAG-His at the BamHI/NotI site. Expression vectors for truncated forms of polydom were also constructed by subcloning of a PCR-amplified cDNA into pSec-His at the BamHI/NotI site or the HindIII/NotI site.

### (2) Expression and Purification of Recombinant Polydom Proteins

Recombinant polydom and its fragments were produced using the Freestyle™ 293 Expression System (Invitrogen). An N-terminal fragment of polydom (hereinafter referred to as “pol-N”; see FIG. 1) is a fragment having the amino acid sequence of residues 18 to 789 of SEQ ID NO: 2 and additionally having a His tag at the C-terminus. A C-terminal fragment of polydom (hereinafter referred to as “pol-C”; see FIG. 1) is a fragment having the amino acid sequence of residues 1192 to 3567 of SEQ ID NO: 2 and additionally having a His tag at the C-terminus, FreeStyle™ 293-F cells were transfected with the expression vectors using 293fectin (Invitrogen) and grown in serum-free Freestyle™ 293 expression medium for 72 hours. The conditioned media were collected and clarified by centrifugation. To check the expression, the cells were lysed with TBS containing 1% (w/v) Nonidet P-40, 1 mM phenylmethylsulfonyl fluoride, 5 μg/ml aprotinin, 5 μg/ml leupeptin and 5 μg/ml pepstatin. The conditioned media and the cell lysates were pulled down using Ni-NTA-agarose (Qiagen) or anti-FLAG M2-agarose (Sigma), and subjected to immunoblotting. For purification of FLAG-tagged proteins, the conditioned media were separately applied to an anti-FLAG M2-agarose column, and the bound proteins were eluted with 100 μg/ml FLAG peptide in PBS. For purification of His-tagged proteins, the conditioned media were subjected to affinity chromatography using Ni-NTA-agarose. After column washing with PBS, the bound proteins were elated with PBS containing 200 mM imidazole. The eluted proteins were dialyzed against PBS. The concentrations of the purified proteins were determined by the Bradford assay using bovine serum albumin (BSA) as a standard.

### (3) Anti-Polydom Antibodies

Antibodies against polydom were raised in rabbits using FLAG-tagged full-length polydom as an immunogen. The antibodies were affinity-purified on columns containing either pol-N or pol-C, The columns were washed with 10 mM Tris-HCl buffer (pH 7.5) containing 0.5 M NaCl, and the bound antibodies were then elated with 0.1 M glycine-HCl (pH 2.8). The elated fractions were immediately neutralized with 1 M Tris-HCl (pH 9.0) and dialyzed against PBS. An Alexa Fluor™ 555-conjugated anti-polydom (pol-N) antibody was prepared using the APEX™ Alexa Fluor antibody labeling kit (Invitrogen).

### (4) Cell Adhesion Assay

A cell adhesion assay was performed as described in Sato, Y. , Uemura, T., Morimitsu, K., Sato-Nishiuchi, R., Manabe, R., Takagi, J., Yamada, M., and Sekiguchi, K. (2009) J. Biol. Chem. 284, 14524-14536. For an assay involving blocking antibodies, RD cells (human rhabdomyosarcoma cells) were preincubated with monoclonal antibodies of interest at room temperature for 15 minutes and plated on 96-well plates coated with recombinant polydom proteins or fibronectin. The cells were incubated for 30 minutes, washed, fixed with 3.7% formaldehyde, and stained with 0.5% toluidine blue.

### (5) Flow Cytometry

The integrin α9β1 expression levels on A549 cells (human lung adenocarcinoma cells), HT1080 cells (human fibrosarcoma cells) and RD cells were verified by flow cytometry using anti-integrin α9β1 monoclonal antibody Y9A2. Normal mouse IgG was used as a control.

### (6) Expression and Purification of Recombinant Integrins

A cDNA encoding the extracellular region of human integrin α9 was amplified by RT-PCR from total RNA extracted from RD cells and cloned into pBluescript KS(+). After verification of the nucleotide sequence, an error-free cDNA fragment was inserted into pcDNA-ACID-FLAG (Nishiuchi, R., Takagi, J., Hayashi, M., Ido, H., Yagi, Y., Sanzen, N., Tsuji, T., Yamada, M., and Sekiguchi, K. (2006) Matrix Biol. 25, 189-197). An expression vector for the extracellular region of integrin β1 was provided by Dr. Junichi Takagi (Takagi, J., Erickson, H. P., and Springer, T. A. (2001) Nat. Struct. Biol. 8, 412-416). The recombinant integrins were produced using the Freestyle™ 293 Expression System (Invitrogen) and purified as described in the above-mentioned publication Matrix Biol. 25, 189-197, (2006).

### (7) SDS-PAGE, Western Blotting and Amino Acid Sequencing

SDS-PAGE was performed according to the Laenmli protocol. The separated proteins were visualized by staining with silver nitrate or Coomassie Brilliant Blue (CBB) or transferred onto a nitrocellulose membrane (for immunoblotting) or a polyvinylidene difluoride membrane (for amino acid sequencing). For immunoblotting, the membrane was probed with antibodies and then visualized with an ECL detection kit (GE Healthcare). For amino acid sequencing, the protein bands on the membrane were visualized by CBB staining and then dissected out. The N-terminal amino acid sequences were analyzed by the method of Edman using a Procise 491 cLC protein sequencer (Applied Biosystems).

### (8) Integrin Binding Assay

An integrin binding assay was performed as described in the above-mentioned publication Matrix Biol. 25, 189-192, (2006). In some experiments, integrins were preincubated with synthetic peptides at various concentrations to evaluate the inhibitory activities of the synthetic peptides. The apparent dissociation constants were determined as described in Nishiuchi, R., Murayama, O., Fujiwara, H., Gu, J., Kawakami. T., Aimoto, S., Wada, Y., and Sekiguchi, K. (2003) J. Biochem, 134, 497-504.

### (2) Expression and Purification of GST-Fused Polydom, Tenascin-C and Osteopontin Fragments

cDNAs encoding CCP21 or its deletion mutants were separately PCR-amplified and subcloned into pGEX4T-1 (GE Healthcare) at the EcoRI/SalI site. CCP21 and the same domain lacking Glu2628-Ser2664 (hereinafter referred to as “ΔD2628-S2664”) were His-tagged at their C-termini. GST fusion proteins were induced in E. coli BL21 by incubation with 0.1 mM IPTG at 25° C. for 2 hours. The cells were lysed by sonication and the supernatants were passed over a glutathione-Sepharose 4B (GE Healthcare) column. The bound proteins were eluted with 50 mM Tris-HCl (pH 8.0) containing 10 mM glutathione. The resulting GST fusion proteins of intact CCP21 or mutant CCP21 were further purified on an Ni-NTA-agarose column. The purified proteins were dialyzed against 20 mM HEPES buffer (pH 8.0) containing 130 mM NaCl and then quantified by the Bradford assay.

A cDNA encoding the third fibronectin type III domain of human tenascin-C (hereinafter referred to as “TNfn3”) was obtained by RT-PCR using RNA extracted from HT1080 cells and subcloned into pBluescript KS(+). A cDNA encoding human osteopontin was purchased from the NIH Mammalian Gene Collection (Invitrogen), A cDNA encoding an osteopontin N-terminal fragment (hereinafter referred to as “OPN-Nhalf”) was PCR-amplified. The RGD motifs within TNfn3 and OPN-Nhalf were then mutated to RAA for specific binding to integrin α9β1 (hereinafter referred to as “TNfn3-RAA” and OPN-Nhalf-RAA,” respectively). After verification of the nucleotide sequence, the cDNA fragments were separately inserted into pGEX4T-1 at the EcoRI/SalI site. Both proteins were expressed and purified as described above.

### (10) Immunohistochemistry

Mouse embryos were embedded in O.C.T. compound (Sakura Finetek) for cryosectioning. The resulting sections were fixed with 3.7% formaldehyde (single staining for polydom) or ice-cold acetone (double staining for polydom and integrin α9). The fixed sections were subjected to pretreatment with chondroitinase ABC (5 U/ml, Seikagaku Corp.) and hyaluronidase (1 U/ml, Sigma) in PBS at 37° C. for 30 minutes, followed by endogenous peroxidase inactivation with 0.3% H₂O₂ and blocking with PBS containing 1% BSA. The sections were labeled with antibodies at 4° C. overnight and washed in PBS. The bound antibodies were visualized with HRP polymer-conjugated anti-rabbit IgG antibody and DAB (Dako), or with Alexa Fluor™-conjugated secondary antibodies. Finally, the sections were counterstained with Mayer's hematoxylin (for DAB staining) or Hoechst 33342 (for immunofluorescence staining), and then mounted with Mount-Quick (Daido Sangyo) or Fluorescent Mounting Medium (Dako).

### (11) In Situ Integrin Binding Assay

Cryosections of mouse embryos were fixed with ice-cold acetone for 15 minutes, washed with TBS, and blocked with 1% BSA in TBS. After three washes with TBS, the sections were incubated with recombinant integrin α9β1 (3 μg/ml) in TBS containing 1% BSA and 1 mM MnCl₂ at 4° C. overnight. The sections were then washed with TBS containing 1 mM MnCl₂ (TBS/Mn) and incubated with an anti-Velcro antibody (0.5 μg/ml) in TBS/Mn containing 1% BSA (TBS/Mn/BSA) at room temperature for 2 hours. After washing with TBS/Mn, the sections were incubated with Alexa Fluor™ 488-conjugated goat anti-rabbit IgG antibody in TBS/Mn/BSA at room temperature for 2 hours. After washing, the sections were incubated with 100 μg/ml normal rabbit IgG (Dako) in TBS/Mn/BSA at room temperature for 1 hour to block secondary antibody unbound sites. After washing with 20 mM HEPES buffer (pH 7.5) containing 130 mM NaCl and 1 mM MnCl₂, the sections were refixed with 3.7% formaldehyde in 20 mM HEPES buffer (pH 7.5) containing 130 mM NaCl and 1 mM MnCl₂ for 10 minutes. The sections were then washed with TBS and incubated with an Alexa Fluor™ 555-conjugated anti-polydom antibody in TBS containing 1% BSA at 4° C. overnight. The sections were washed with TBS and mounted with Fluorescent Mounting Medium (Dako).

### (12) Antibodies Used

A mouse monoclonal antibody (mAb) against human integrin α3 (3G8) and a mouse mAb against human integrin α3 (8F1) were raised in the inventors' laboratory as described in Kikkawa, Y., Sanzen, N., Fujiwara, H., Sonnenberg, A., and Sekiguchi, K. (2000) J. Cell Sci. 113, 869-876; and Manabe, K,, Obe, N., Maeda, T., Fukuda, T., and Sekiguchi, K. (1997) J. Cell Biol. 139, 295-307, respectively. An mAb against integrin α6 subunit (AMCI7-4) was provided by Dr. Masahiko Katayama (Tsukuba Research Laboratories, Eisai Co.) (Katayama, M., Sanzen, N., Funakoshi, A., and Sekiguchi, K. (2003) Cancer Res. 63, 222-229), A mouse mAb against human integrin β1 (AIIB2) was obtained from the Developmental Studies Hybridoma Bank (Iowa). A mouse mAb against human integrin α2 subunit (P1E6), a mouse mAb against human integrin 660 9β1 (Y9A2), and normal mouse IgG were purchased from Santa Cruz Biotechnology. Inc. A goat polyclonal antibody against mouse integrin α9 was obtained from R&D Systems. An HRP-conjugated anti-FLAG M2 antibody and an anti-penta-His antibody were obtained from Sigma and Qiagen, respectively. An anti-Velcro antibody was raised in rabbits by immunization with coiled-coil ACID and BASE peptides as described in Takagi, J., Erickson, H. P., and Springer, T. A. (2001) Nat. Struct. Biol. 8, 412-416.

### Experimental Results

### (I) Confirmation of Recombinant Polydom

The spent culture medium of cells for the expression of a recombinant polydom having the signal sequence of the mouse immunoglobulin κ chain was affinity-purified using an anti-FLAG monoclonal antibody, and the resulting polydom was subjected to 6% SDS-PAGE under non-reducing or reducing conditions.

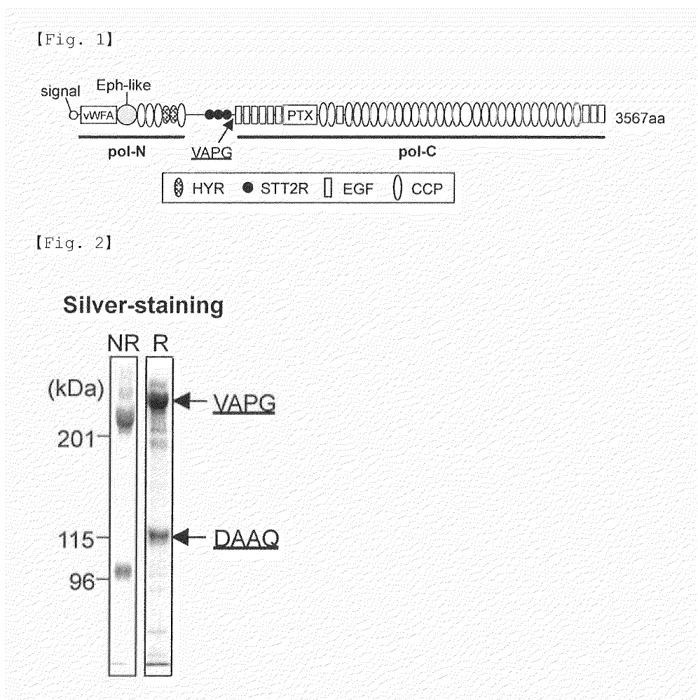

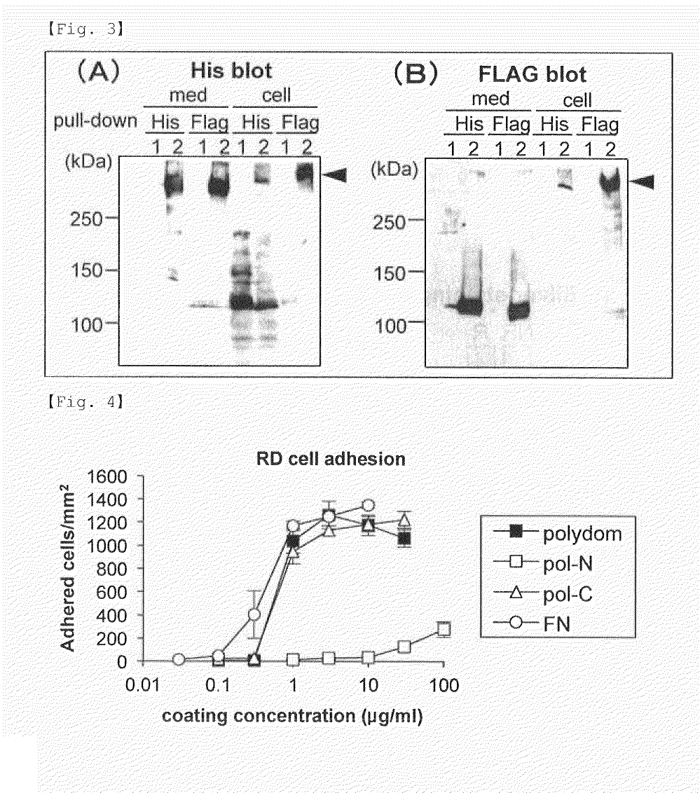

The results are shown in FIG. 2. As is clear from FIG. 2, two major bands of 220 kDa and 100 kDa were detected under non-reducing conditions (NR). A similar banding pattern was obtained under reducing conditions (R) except that the corresponding two major bands migrated more slowly (270 kDa and 115 kDa), suggesting that both were enriched in intrapeptide disulfide bonds. Determination of the N-terminal amino acid sequences of the two protein bands revealed that the 115 kDa band started with DAAQ, which is the putative N-terminal sequence lacking the signal sequence of the mouse immunoglobulin κ chain. It was also revealed that the 270 kDa band started with VAPG, which is identical to the sequence of residues 1177 to 1180 of SEQ ID NO: 2 located N-terminal to the first EGF domain. These findings indicate that polydom was proteolytically processed, before or after secretion, into two parts (i.e. the N-terminal 115-kDa fragment and the C-terminal 270-kDa fragment), which remained associated with each other after the proteolytic cleavage.

Next, a recombinant polydom having an N-terminal FLAG tag and a C-terminal His tag was expressed in 293-F cells and then precipitated from the spent medium and cell lysate with anti-FLAG antibody beads or Ni-NTA beads. The precipitates were subjected to 5% SDS-PAGE under reducing conditions and analyzed by immunoblotting using an anti-His antibody and an anti-FLAG antibody. Similar procedures were performed on untransfected 293-F cells as a negative control.

The results are shown in FIGS. 3(A) and (B). (A) shows the results of immunoblotting using an anti-His antibody, and (B) shows the results of immunoblotting using an anti-FLAG antibody. In FIG. 3, med represents medium, cell represents cell lysate, and His represents the precipitate obtained using Ni-NTA beads, and FLAG represents the precipitate obtained using anti-FLAG antibody beads. Lane 1 is for the negative control and lane 2 is for polydom, In the precipitate from the spent medium, both the 270-kDa and 115-kDa fragments were recovered irrespective of the type of bead used, thus confirming that these fragments remained associated with each other. Notably, a high molecular weight band migrating in the >300 kDa region was detected in the precipitate from the cell lysate irrespective of the type of bead used (arrowheads in FIGS. 3(A) and (B)). Therefore, it seems likely that the proteolytic processing of polydom into the N-terminal 115-kDa and C-terminal 270-kDa fragments occurs after secretion.

(2) Polydom Mediates Integrin α9β1-dependent Cell Adhesion

Whether polydom is capable of promoting cell adhesion was examined with the use of the following three human cell lines: A549 human lung adenocarcinoma cells, HT1080 human fibrosarcoma cells, and RD human rhabdomyosarcoma cells. The cells were plated on 96-well plates coated with serial diluted concentrations of full-length polydom, pol-N, pol-C or plasma fibronectin (FN) and incubated at 37° C. for 30 minutes. After washing out unattached cells, attached cells were fixed and stained with toluidine blue. The attached cells were counted under a microscope. The assay was performed in triplicate.

The results of RD cells are shown in FIG. 4. As is clear from FIG. 4, RD cells adhered to full-length polydom in a coating concentration-dependent manner, attaining the maximum adhesion at a coating concentration of 3 μg/ml. In the coating with pol-N or pol-C, only pol-C was capable of promoting cell adhesion, with a potency that was almost equivalent to that of full-length polydom. Although data are not shown, A549 and HT1080 cells did not adhere to polydom.

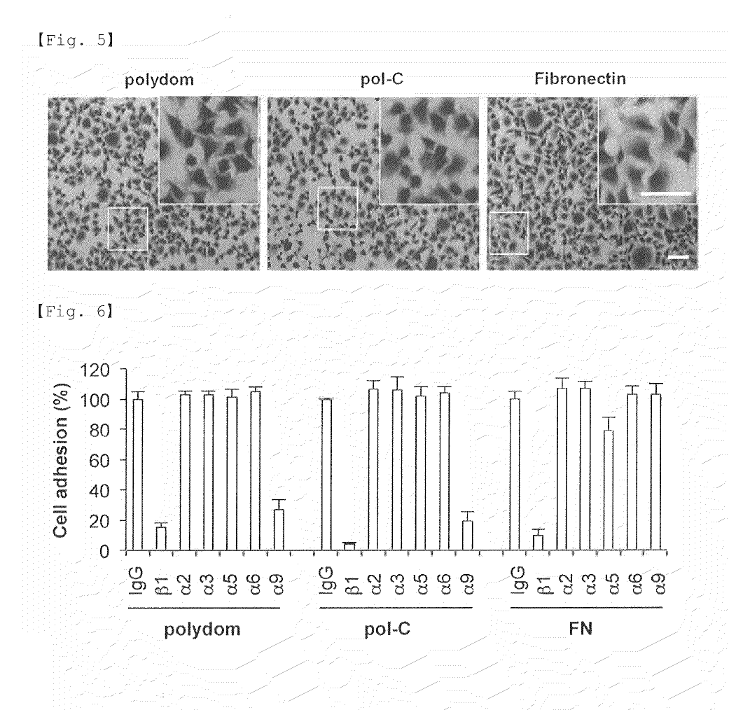

FIG. 5 shows the results of microscopic observation of adhesion of RD cells to full-length polydom, pol-C and fibronectin. As is understood from FIG. 5, the cells attached onto polydom or pol-C exhibited spread morphology, although the extent of the spreading was less pronounced than that of the cells attached onto fibronectin.

These findings indicate that polydom is capable of mediating cell adhesion and subsequent cell spreading, and that the cell adhesion-promoting activity resides within the C-terminal region composed of an array of CCP domains.

Next, it was explored whether the adhesion of RD cells to polydom is dependent on integrins, which are major cell surface receptors for ECM proteins. Ninety-six-well plates were coated with full-length polydom (3 μg/ml) , pol-C (3 μg/ml) or plasma fibronectin (1 μg/ml). RD cells were preincubated with seven kinds of function-blocking mAbs (10 μg/ml) at room temperature for 10 minutes and then added to the precoated wells. The seven kinds of function-blocking mAbs were control mouse IgG (IgG), an anti-integrin β1 mAb AIIB2 (β1), an anti-integrin α2 mAb P1E6 (α2), an anti-integrin α3 mAb 3G8 (α3), an anti-integrin α5 mAb 8F1 (α5), an anti-integrin α6 mAb GoH3 (α6) and an anti-integrin α9β1 mAb Y9A2 (α9). After 30 minutes of incubation at 37° C., unattached cells were washed out. Then, attached cells were fixed and stained with toluidine blue.

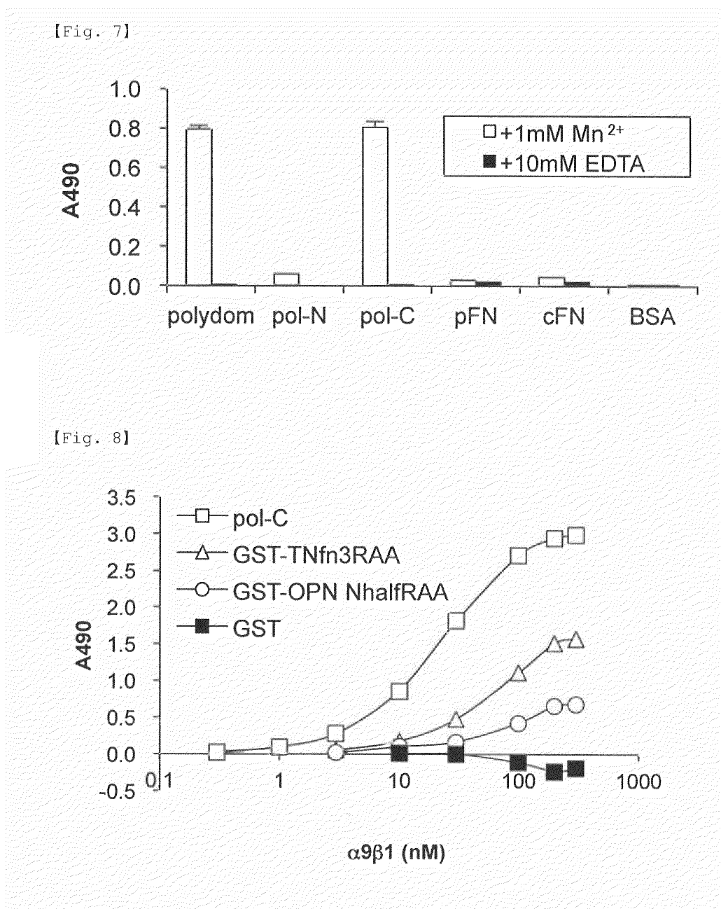

The results are shown in FIG. 6. In the figure, the number of the attached cells is expressed as a percentage relative to the number of the attached cells in the treatment with control mouse IgG (100%). As is clear from FIG. 6, the mAb against integrin β1 subunit strongly inhibited the adhesion of RD cells to polydom. The mAbs against integrin α2, α2, α3, α5 and α6 subunits did not inhibit the adhesion of RD cells to polydom, whereas the mAb against integrin α9β1 strongly inhibited the adhesion of RD cells to polydom. These findings are consistent with the finding that A549 and HT1080 cells did not adhere to polydom because integrin α9β1 was expressed on RD cells but not on A549 or HT1080 cells. Thus, it seems likely that the cell adhesion to polydom is primarily mediated by integrin α9β1.

### (3) Polydom is a Preferred Ligand for Integrin α9β1

To corroborate the role of integrin α9β1 as an adhesion receptor for polydom, a direct integrin binding assay was performed, using recombinant integrin α9β1. Plates were coated with full-length polydom, pol-N, pol-C, plasma fibronectin (pFN) or cellular fibronectin (cFN), and recombinant integrin α9β1 was added and allowed to bind thereto in the presence of 1 mM MnCl₂ or 10 mM EDTA. BSA was used as a negative control. The bound integrin was quantified using a biotinylated anti-Velcro antibody and HRP-conjugated streptavidin.

The results are shown in FIG. 7. As is clear from FIG. 7, the recombinant integrin α9β1 bound to full-length polydom and pol-C, but not to pol-N. The binding of the recombinant integrin α9β1 to polydom was completely abrogated in the presence of EDTA, thus confirming the specificity of the integrin binding assay. Although cellular fibronectin, which contains the EDTA domain, is a putative ligand for integrin α9β1, the recombinant integrin α9β1 exhibited only marginal binding activity for cellular fibronectin. These findings are consistent with those of the ceil adhesion assay and corroborate the notion that integrin α9β1 binds directly to polydom, which has the integrin-binding site in the C-terminal 270-kDa region.

Integrin α9β1 is known to bind to the third fibronectin type III domain of tenascin-C (TNfn3) and the osteopontin N-terminal half (OPN-Nhalf). These integrin α9β1 ligands were recombinantly expressed as GST fusion proteins and then purified. In the GST fusion proteins, the RGD cell-adhesive motifs were replaced with RAA to nullify their abilities to interact with RGD-binding integrins, such as those containing the αv subunit, Microtiter plates were coated with pol-C (10 nM), GST-TNfn3-RAA (100 nM) or GST-OPN-Nhalf-RAA (100 nM), and integrin α9β1 was added and allowed to bind thereto in the presence of 1 mM MnCl₂.

The results are shown in FIG. 8. As is clear from FIG. 8, TNfn3-RAA and OPN-Nhalf-RAA were capable of binding to integrin α9β1, but their affinities for integrin α9β1 were significantly lower than that of pol-C. The apparent dissociation constants for these integrin α9β1 ligands were unable to be determined due to incomplete saturation of binding at the highest integrin concentration, whereas the dissociation constant between integrin α9β1 and pol-C was estimated from three independent determinations to be 32.4±2.7 nM.

### (4) Integrin α9β1 Binds to CCP21

To locate the integrin α9β1-binding site within polydom, a series of five N-terminal deletion mutants of polydom (pol-C, ΔEGF6, ΔPTX, ΔCCP20 and ΔCCP21; see FIG. 9) were constructed. Their binding activities for integrin α9β1 were evaluated in the presence of 1 mM MnCl₂ or 10 mM EDTA using microtiter plates coated with these recombinant proteins (10 nM).

The results are shown in FIG. 10, As is clear from FIG. 10, although deletion of the region upstream of CCP21 did not compromise the integrin binding activity of pol-C, deletion of CCP21 resulted in a dramatic loss of the integrin binding activity, thus underscoring the critical role of CCP21 in the integrin binding activity. To explore whether CCP21 harbors the integrin α9β1-binding site, recombinant CCP21 as a GST fusion protein was produced and examined for the integrin α9β1-binding activity. As shown in FIG. 10, CCP21 alone was fully active in binding to integrin α9β1, thus confirming the critical role of CCP21 in the integrin binding activity.

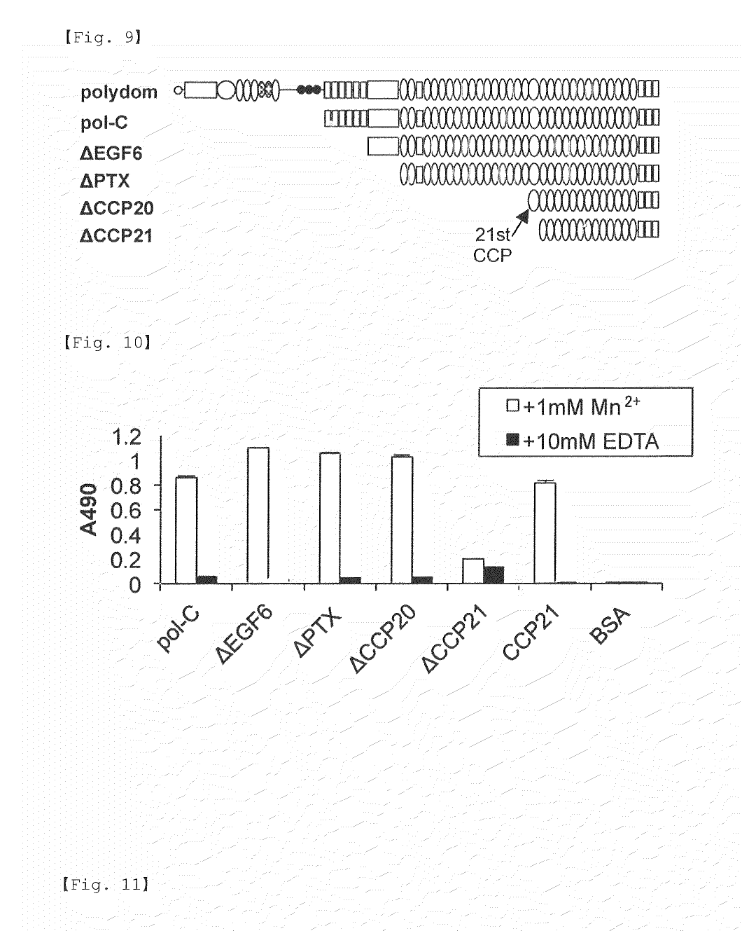

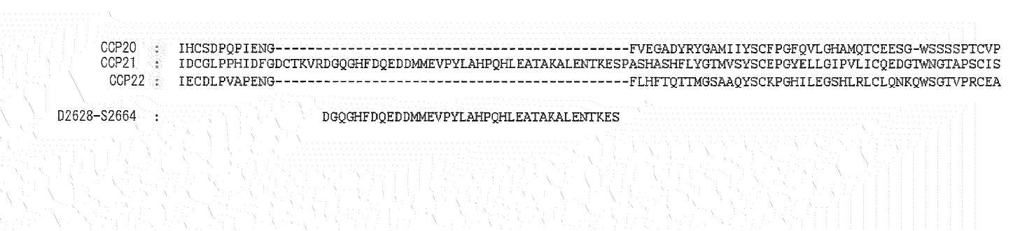

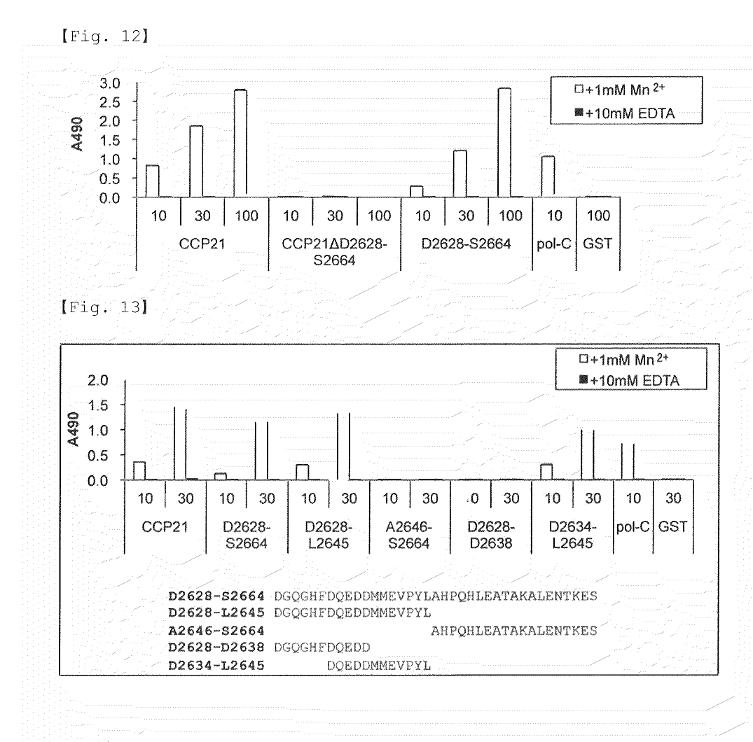

FIG. 11 shows the alignment of the amino acid sequences of the 20th, 21st and 22nd CCP domains by ClustalW. Among the 34 CCP domains within polydom, CCP21 is unique because it contains about 40 extra amino acids as compared with the other CCP domains. An assay to examine whether this extra region (hereinafter referred to as “extra segment”) within CCP21 is involved in binding to integrin α9β1 was performed, Microtiter plates were coated with a GST-CCP21 fusion protein, a D2628-S2664 deletion mutant of the GST-CCP21 fusion protein (CCP21ΔD2628-S2664), D2628-S2664 alone (see FIG. 11), pol-C or GST alone, and integrin α9β1 was added and allowed to bind thereto in the presence of 1 mM MnCl₂ or 10 mM EDTA.

The results are shown in FIG. 12. As is clear from FIG. 12, CCP21ΔD2628-S2664 was unable to bind to integrin α9β1, whereas D2628-S2664 retained the binding activity for integrin α9β1. These findings show that the integrin α9β1-binding site of polydom is located within the extra 37-amino acid segment (D2628-S2664) of CCP21.

### (5) Integrin α9β1 Recognizes the Sequence EDDMMEVPY

To further narrow down the region responsible for binding to integrin α9β1, the extra 37-amino acid segment of CCP21 was divided into smaller segments, of which GST fusion proteins were produced. Microtiter plates were coated with eight kinds of segments including CCP21, pol-C and GST alone, and integrin α9β1 was added and allowed to bind thereto in the presence of 1 mM MnCl₂ or 10 mM EDTA.

The results are shown in FIG. 13. As is clear from FIG. 13, integrin α9β1 bound only to D2628-L2645 and D2634-L2645, which were within the N-terminal region of D2628-S2664. These findings show that the integrin α9β1-binding site can be mapped to the 12-amino acid segment (D2634-L2645) in the extra segment of CCP21.

To identify the residues involved in binding to Integrin α9β1, alanine scanning mutants and N-terminal or C-terminal deletion mutants of D2634-L2645 were produced as GST fusion proteins. Microtiter plates were coated with the GST fusion proteins containing the mutated segments and integrin α9β1 was added and allowed to bind thereto.

The alanine scanning mutants are shown in FIG. 14. As is clear from FIG. 14, alanine substitution of Glu2641 (E2641A) almost completely abrogated the integrin binding activity, whereas alanine mutations at the residues from Glu2636 to Tyr2644 caused partial reductions in the integrin binding activity to variable extents. These findings indicate that integrin α9β1 recognizes the EDDMMEVPY sequence, within which Glu2641 is the critical acidic residue involved in ligand recognition of integrin α9β1.

To further corroborate the role of EDDMMEVPY as the integrin α9β1 recognition sequence, an integrin α9β1-pol-C binding inhibition assay was performed using synthetic peptides consisting of the EDDMMEVPY sequence or a part of the sequence, Integrin α9β1 (10 nM) was incubated on microtiter plates coated with pol-C (10 nM) in the presence of 1 mM MnCl₂ and various concentrations of the synthetic peptides. To prevent precipitation of the peptides, the assay was performed in the presence of 10% DMSO. Based on the amount of integrin α9β1 bound to pol-C set as 100%, the relative binding amount was calculated.

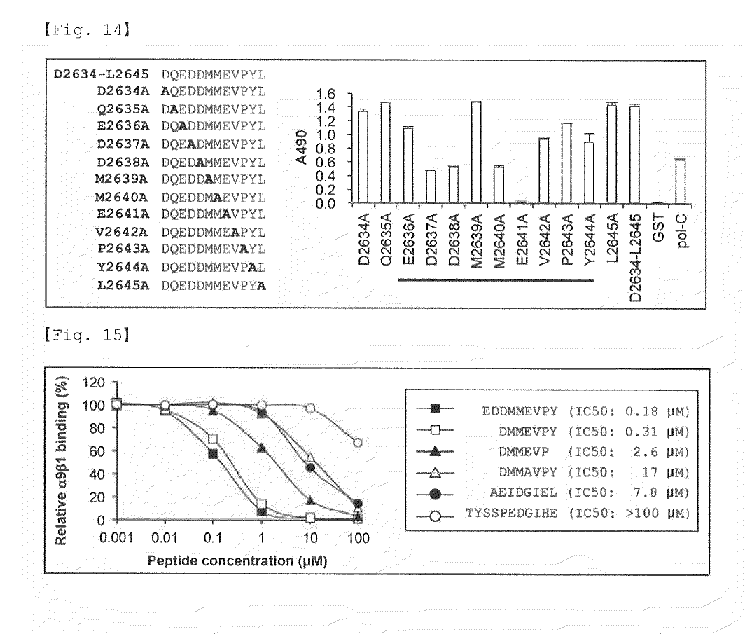

The results are shown in FIG. 15. As is clear from FIG. 15, the EDDMMEVPY peptide inhibited the binding of integrin α9β1 to pol-C in a dose-dependent manner and had an IC50 of 0.18 μM. A smaller peptide, DMMEVPY, was nearly as potent as EDDMMEVPY in inhibiting integrin α9β1 binding, and this finding is consistent with the relatively small contribution of the N-terminal Glu—Asp residues to the binding to integrin α9β1. Further, deletion of the C-terminal Tyr residue from the DMMEVPY peptide resulted in reduction in the inhibitory potency, thus supporting the involvement of Tyr2644 in the CCP21 recognition by integrin α9β1. Alanine substitution of Glu2641 in the DMMEVPY peptide resulted in a marked, reduction in the inhibitory potency, and this finding is consistent with the importance of Glu2641 in the CCP21 recognition of integrin α9β1.

Further, the inhibitory activity of the EDDMMEVPY peptide was compared with those of the peptides AEIDGIEL and TYSSPEDGIME, which represent the integrin α9β1 recognition sequences in tenascin-C and the fibronectin EIIIA domain, respectively. The results are shown in FIG. 15. As is clear from FIG. 15, the AEIDGIEL peptide was capable of inhibiting the binding of integrin α9β1 to pol-C and had an IC50 of 7.8 μM, although its potency was one order of magnitude less than that of the EDDMMEVPY peptide. The TYSSPEDGIHE peptide was barely inhibitory toward the binding of integrin α9β1 to pol-C. These findings support the conclusion that integrin α9β1 binds to polydom with an affinity that is significantly higher than those to tenascin-C and other known integrin α9β1 ligands, and that EDDMMEVPY is a preferred sequence for ligand recognition of integrin α9β1.

(6) Polydom Partially Colocalizes with Integrin α9 in Tissues

The relatively high binding affinity of polydom for integrin α9β1 suggests that polydom functions as a physiological ligand for integrin α9β1. To address this possibility, immunofluorescence staining of mouse embryonic tissues with antibodies against polydom and integrin α9 was performed.

The results are shown in FIG. 16. Polydom was colocalized with integrin α9 at the submucosal mesenchymes in the stomach and intestine (FIG. 16, A to F) . Integrin α9 was expressed in the smooth muscle layers in the stomach and intestine as well, where polydom was barely expressed. Polydom and integrin α9 were also colocalized at the sinusoids in the liver (FIG. 16, G to I), and at Bowman's capsules and the mesenchyme between renal tubules in the kidney (FIG. 16, J to L). In the lung, polydom was detected in the mesenchyme, where integrin α9 was only partially colocalized with polydom (FIG. 16, M to O), Integrin α9 was strongly expressed in the smooth muscle layer of the lung, where polydom was barely detectable. These findings demonstrate that polydom was colocalized with integrin α9 in the embryonic mesenchymes of various organs, except for the smooth muscle layers, where integrin α9 was highly expressed, and are consistent with the possibility that polydom serves as one of the physiological ligands for integrin α9.

To further corroborate the role of polydom as a physiological ligand for integrin α9β1, an in situ integrin binding assay was performed for visualization of integrin α9β1 ligands in whole tissues.

The results are shown in FIG. 17. As shown in A, E and I of FIG. 17, in the cryosections of mouse embryos incubated with integrin α9β1 in the presence of Mn²⁺, its ligands were detected in the mesenchymes and smooth muscle layers of the stomach, intestine and lung, As shown in D, H and L of FIG. 17, no signals were detected when the assay was performed in the presence of EDTA, thus confirming the specificity of the in situ integrin binding assay. As shown in B, F and J of FIG. 17, polydom was detected by immunofluorescence staining using an Alexa Fluor™ 555-conjugated anti-polydom antibody, and as shown in C, G and E in FIG. 17, the signals for polydom overlapped with those for bound integrin α9β1 in the mesenchymal regions of the stomach, intestine and lung. These findings support the conclusion that polydom serves as a physiological ligand for integrin α9β1 of mesenchymal ECM in these organs.

### Example 2

### Interaction of Novel Ligand for Integrin α9β1 with Hematopoietic Stem Cells

Bone marrow cells were flushed out from the femurs and tibias of three mice of 6 to 7 weeks of age (C57BL/6J) and dissociated with a 22G needle. The cells were reacted with a biotin-labeled anti-lineage antibody for 30 minutes and subsequently with an anti-blotin antibody-conjugated magnetic beads, and lineage-positive cells were removed by automated magnetic cell sorter autoMACS (Miltenyi Biotec). After staining with PE-CD3, PE-Mac1, PE-Gr1, PE-Ter119, PE-B220, FTTC-Sca1 and APC-c-Kit, a lineage(−) Sca1(+) c-Kit (high) cell fraction was sorted out using FACSAria and used for the experiments described below.

### (1) Examination of Proliferation and Differentiation of Hematopoietic Stem Cells

The above-prepared cells were plated on a 48-well plate coated with a recombinant polydom protein or fibronectin. The same type of plate without coating was used as a control. The medium used was RPMI containing 10% fetal calf serum, thrombopoietin (30 ng/ml) and flt3 ligand (100 ng/ml). For the examination of cell proliferation, the cells were plated in duplicate wells for each coating at 1000 cells/well and started to be cultured, and the cell number was counted at the start of culture, and on day 2 and day 3. The remaining cells were divided into three aliquots, plated in wells for each coating, and subjected to FACS analysis on day 3 after the start of culture. For the FACS analysis, staining with an antibody cocktail containing PE-CD3, PE-Mac1, PE-Gr1, PE-Ter119, PE-B220, FITC-Sca1 and APC-c-Kit was performed and FACSCont was used.

The results of the examination of cell proliferation are shown in FIG. 18. As is clear from FIG. 18, the cells on the fibronectin-coated wells proliferated as with the control, whereas the cells on the polydom-coated wells were remarkably inhibited from proliferating.

The results of the FACS analysis are shown in FIG. 19. In FIG. 19, the vertical axis represents the expression amount of c-Kit, and the horizontal axis represents the expression amount of Sca1. As is clear from FIG. 19, the cells on the polydom-coated well maintained the expression of Sca1, but had reduced expression of c-Kit, a growth factor receptor. Meanwhile, the cells on the fibronectin-coated well had increased expression of c-Kit.

The above findings indicate that, by interaction with polydom, hematopoietic stem cells were inhibited from proliferating and maintained in their undifferentiated state.

(2) Examination of Effects of EDDMMEVPY Peptide on Interaction of Hematopoietic Stem cells with Polydom Protein

The above-prepared cells were divided into three groups and plated on 48-well plates at 5000 cells/well. The medium used was RPMI containing 10% fetal calf serum, thrombopoietin (30 ng/ml) and flt3 ligand (100 ng/ml). For an EDDMMEVPY peptide treatment group, 2 μl of the peptide was added to each well at the final concentration of 50 μM, and for the other two groups, 2 μl of PBS was added to each well. Then, the culture was performed at 31° C. for 2 hours. Subsequently, the cells were transferred to 48-well plates coated with a recombinant polydom protein or fibronectin. The cells cultured in the presence of the peptide were transferred to the recombinant polydom protein-coated plate, and the cells in one of the other two groups were transferred to the recombinant polydom protein-coated plate, and the cells in the remaining group were transferred to the fibronectin-coated plate. The culture was continued for 3 days and the cell numbers were counted on day 2, day 3 and day 4.

The results are shown in FIG. 20. As is the case with FIG. 18, the cells on the fibronectin-coated plate (in the figure, Fibronectin) proliferated, whereas the cells on the polydom-coated plate (in the figure, Polydom) were remarkably inhibited from proliferating. Meanwhile, the cells cultured on the polydom-coated plate after treatment with the EDDMMEVPY peptide (in the figure, Polydom+peptide 50 μM) were shown to proliferate albeit more slowly than the cells on the fibronectin-coated plate. This result is probably attributed to the inhibition of the interaction of integrin α9β1 on the hematopoietic stem cells with polydom on the plate by the binding of the EDDMMEVPY peptide to integrin α9β1 on the hematopoietic stem cells.

### Example 3

### Interaction of Novel Ligand for Integrin α9β1 with Cardiac Stem Cells

### (1) Separation of Cardiac Stem Cells

Tissue stem cells are known to generally have a very prolonged, cell cycle and thus allow labeling compounds incorporated into DNA and the cell interior to remain stable for a long period of time without decay along with cell division, This nature can be advantageously used to identify and separate tissue stem cells by short-term labeling or cells with a nucleotide analog such as bromodeoxyuridine (BrdU) or a fusion protein of green fluorescent protein (hereinafter referred to as “GFP”) and a nucleoprotein, histone, followed by selection of cells retaining the label for a long period of time (label-retaining cells). In this experiment, with the use of a tetracycline-inducible expression system in combination with the ROSA26 promoter, a fusion protein of GFP and histone H2B (H2B-GFP) was expressed in the heart of a mouse for 2 weeks starting 1 week before birth, and 6 weeks later, cardiac stem cells were separated as GFP label-retaining cells from the heart.

### (2) Analysis of Integrin α9 Expression in GFP Label-Retaining Cardiac Cells

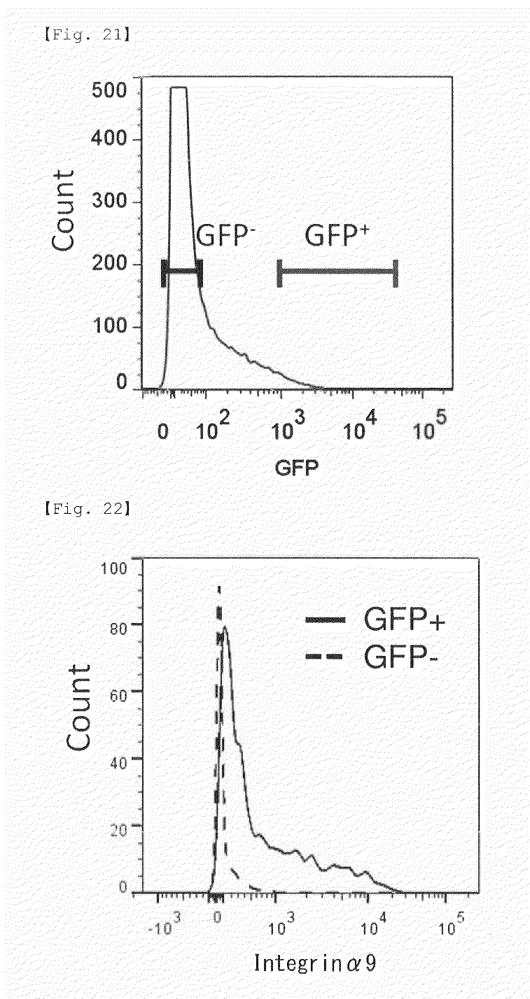

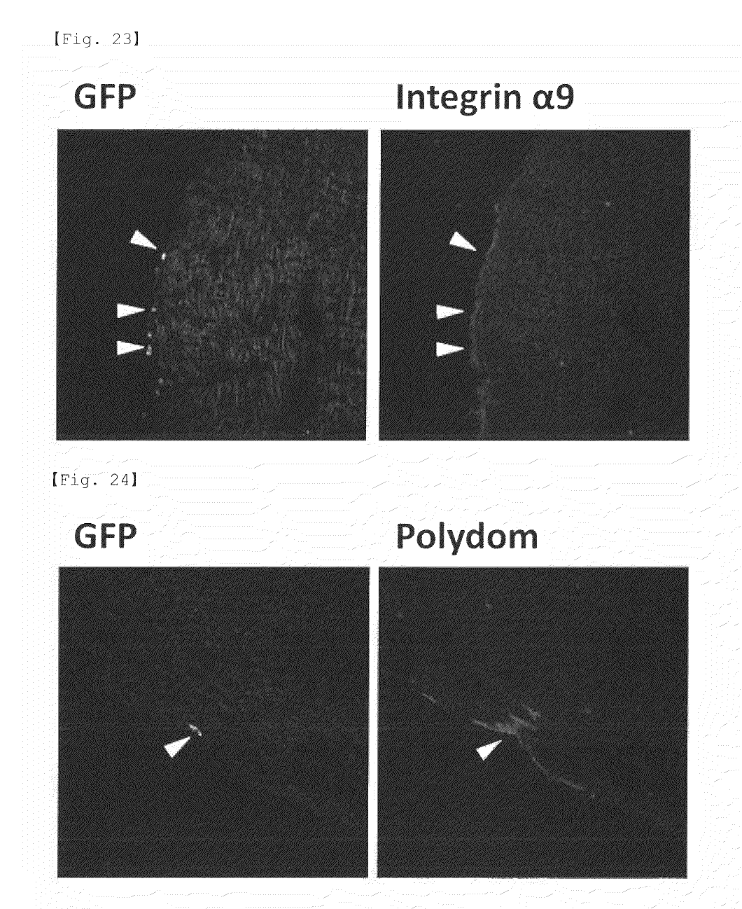

H2B-GFP was forcibly expressed in the heart of a mouse for 2 weeks around birth and then chased for 6 weeks, and GFP label-retaining cells were prepared from the heart. The cardiac cells were treated at 37° C. for 30 minutes with Hank's Balanced Salt Solution (hereinafter referred to as “HBSS”) containing 0.1% collagenase B (Roche) and 2.4 U/ml dispase (Life Technologies) for dissociation, and then passed through a BD Falcon cell strainer to give single cells. The resulting cells were fractionated based on the fluorescence of GFP using a cell sorter FACSAria (manufactured by BD) to separate GFP-positive cells (see FIG. 21). GFP-positive cells and GFP-non-positive cells were separately suspended at 10⁶ cells in 100 μl of HBSS containing 1 μg of an anti-mouse integrin α9 antibody (R&D) and 1% BSA, and reacted with the antibody on ice for 30 minutes. After washing with 500 μl of HBSS containing 1% BSA, the ceils were suspended in 100 μl of HBSS containing an allophycocyanin-labeled anti-goat IgG antibody (R&D, 20-fold dilution) and 1% BSA, and reacted with the antibody on ice for 20 minutes to fluorescently label integrin α9 on the cells. After washing with 500 μl of HBSS containing 1% BSA, the ceils were resuspended in 500 μl of HBSS containing 1% BSA and analyzed for the expression of integrin α9 using a cell sorter FACSAria, As shown in FIG. 22, the cells highly expressing integrin α9 were concentrated in the GFP-positive cell population.

### (3) Immunohistochemical Staining of Integrin α9

To confirm the high expression of integrin α9 on cardiac stem cells, GFP label-retaining cells and integrin α9 expressing cells were analyzed by double immunofluorescence staining. A mouse heart containing H2B-GFP-labeled cardiac stem cells was cryosectioned at a thickness of 8 μm and the resulting sections were fixed with 3.7% formalin for 10 minutes, After blocking with PBS containing 3% BSA at room temperature for 30 minutes, the sections were reacted with an anti-mouse integrin α9 antibody (5 μg/ml) at 4° C. overnight and the bound antibody was then fluorescently labeled with an Alexa Fluor™ 546-labeled anti-goat IgG antibody. The fluorescently-labeled integrin α9 was observed with a confocal microscope, GFP label-retaining cells were identified as cardiac stem cells.

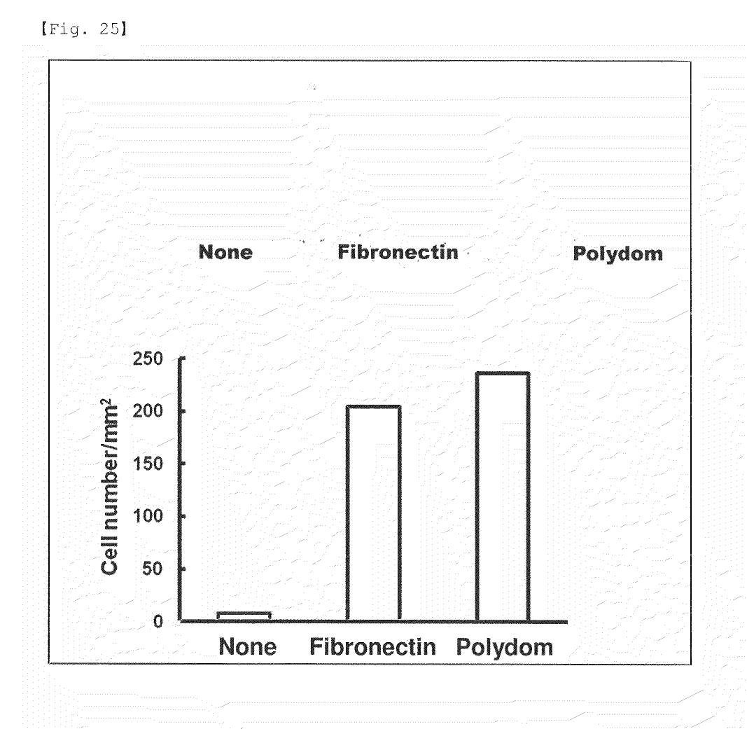

The results are shown in FIG. 23. The left panel is a GFP-stained image, the right panel is an integrin α9-stained image, and the arrowheads in the figure represent the locations of GFP label-retaining cells. FIG. 23 shows that the GFP label-retaining cells were mainly localized in the epicardium and colocalized with the integrin α9 expressing cells.

### (4) Immunohistochemical Staining of Polydom

To explore whether polydom, an integrin α9 ligand, is expressed around cardiac stem cells, double immunofluorescence staining was performed in the same manner as in the above (3). The concentration of the anti-polydom antibody used was 2 μg/ml, and the bound anti-polydom antibody was visualized with an Alexa Fluor™ 546-labeled anti-rabbit IgG antibody.

The results are shown in FIG. 24. The left panel is a GFP-stained image, the right panel is a polydom-stained image, and the arrowheads in the figure represent the locations of GFP label-retaining cells. As shown in FIG. 24, the expression of polydom was localized around the GFP label-retaining cells in the epicardium. These findings support the possibility that polydom functions as a ligand for cardiac stem cell-expressed integrin α9β1.

### (5) Cell Adhesion Assay Using GFP Label-Retaining Cells

To explore whether polydom serves as a scaffold for integrin α9β1-expressing cardiac stem cells, cardiac stem cells were separated as GFP label-retaining cells and subjected to a cell adhesion assay. A 96-well immuno plate (Nunc Maxisorp) was coated with 50 μl/well of 100 nM polydom or fibronectin at 4° C. overnight, GFP label-retaining cells were collected by FACSAria and suspended at 1×10⁶ cells/ml in serum-free Iscove's Modified Dulbecco Medium (hereinafter referred to as “IMDM”). The cell suspension was plated at 100 μl/well and cultured in a 5% CO₂ incubator at 37° C. for 12 hours. After three washes with PBS, the cells were fixed with 100 μl/well of 3.7% formalin for 15 minutes. Then, the cells were incubated with 0.5% toluidine blue (150 μl/well) for 10 minutes and washed with MilliQ water. After drying, the number of attached cells was counted under a microscope.

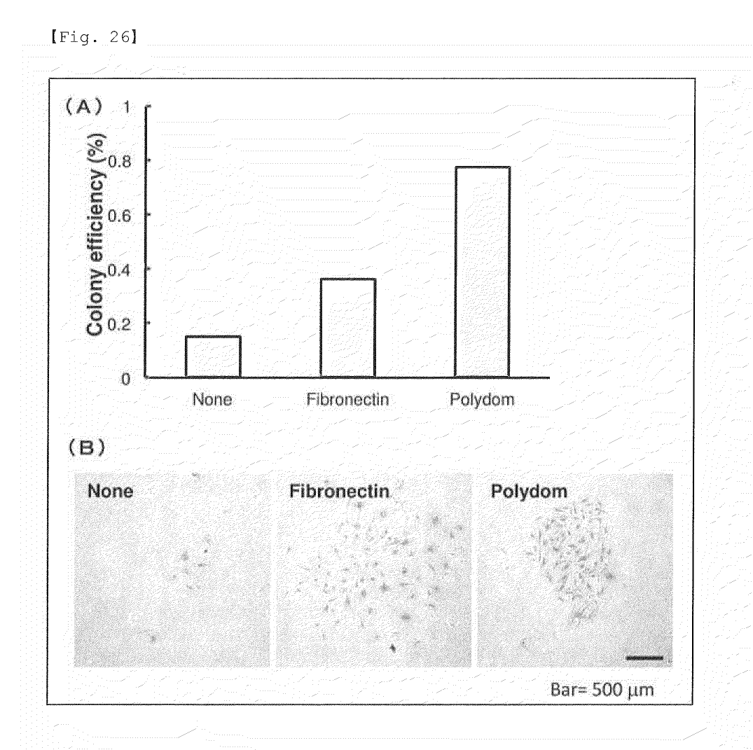

The results are shown in FIG. 25. The upper panel is a set of microscopic images showing attached cells, and the lower panel is a graph showing the cell adhesion activity (the number of attached cells per square millimeter). As is clear from FIG. 25, the cells hardly adhered onto the uncoated plate, whereas a large number of the cells adhered onto the fibronectin-coated plate and the polydom-coated plate. It was found that polydom is equivalent or superior to fibronectin in cell adhesion activity for cardiac stem cells.

### (6) Measurement of Colony Forming Ability Using GFP Label-Retaining Cells

To explore whether cardiac stem cells proliferate on a polydom-coated plate, colony formation was measured, as an indicator of proliferation. A Falcon 6-well plate was coated with 800 μl/well of 100 nM polydom or fibronectin at 4° C. overnight. GFP label-retaining cells were collected by FACSAria and suspended in IMDM containing 10% fetal calf serum. The cells were plated at 1×10⁴ cells/well and cultured in a 53 CO₂ incubator at 37° C. The culture was continued for 2 weeks with medium replacement every three days. After washing with HBSS containing 1 mM CaCl₂, Giemsa stain (Merck) was added at 1 ml/well, and 30 minutes later, washing with tap water was performed. After drying, the number of colonies was counted under a microscope.

The results are shown in FIG. 26. (A) is a graph showing the colony forming activity and (B) is a set of images showing typical colony morphologies. The colony forming ability was expressed as the percentage of the colony count relative to the number of the cells plated. As is clear from FIG. 26, the colony forming activity of the cells on the uncoated plate was 0.2% or less, whereas the colony forming activity of the cells on the polydom-coated plate was about 0.8%. The colony forming activity of the cells on the fibronectin-coated plate was about 0.4%. That is, it was shown that cardiac stem cells on polydom are more capable of forming colonies than those on fibronectin.

The present invention is not limited to particular embodiments and examples described above, and various modifications can be made within the scope of the appended claims. Other embodiments provided by suitably combining technical means disclosed in separate embodiments of the present invention are also within the technical scope of the present invention. All the academic publications and patent literatures cited in the above description are incorporated herein by reference.

1-12. (canceled)

13. A ligand for integrin α9β1, consisting of a peptide having the following amino acid sequence:
(A) EDDMMEVPY (SEQ ID NO: 1) or
(B) an amino acid sequence the same as the amino acid sequence represented by SEQ ID NO: 1 except for having deletion, substitution or addition of 1 to 3 amino acids.

14. The ligand for integrin α9β1 according to claim 13, wherein the ligand is SVEP1 or an active fragment thereof.

15. The ligand for integrin α9β1 according to claim 13, wherein the ligand has 2500 amino acid residues or less.

16. A polynucleotide encoding a peptide constituting the ligand for integrin α9β1 according to claim 13.

17. An expression vector containing the polynucleotide according to claim 16.

18. An antibody against the ligand for integrin α9β1 according to claim 13.

19. A method for screening for substances capable of inhibiting interaction of SVEP1 with integrin α9β1, the method using the ligand for integrin α9β1 according to claim 13.

20. A method for culturing integrin α9β1-expressing cells, the method using the ligand for integrin α9β1 according to claim 13 as a substrate.

21. A method for culturing tissue stem cells with maintenance of their undifferentiated state and inhibition of their proliferation, the method using the ligand for integrin α9β1 according to claim 13 as a substrate.

22. A method for separating integrin α9β1-expressing cells, the method allowing selective separation of cells capable of binding to the ligand for integrin α9β1 according to claim 13.

23. A drug delivery system for delivering a drug to integrin α9β1-expressing cells, the system comprising the ligand for integrin α9β1 according to claim 13, and the drug.

24. A method for inducing tissue stem cells in a resting phase or an antiproliferative state into a proliferative state, the method comprising bringing the stem cells into contact with the ligand for integrin α9β1 according to claim 13.

25. The ligand for integrin α9β1 according to claim 13, wherein the ligand is an active fragment of SVEP1.

26. A ligand for integrin α9β1 consisting of the following peptide:
(a) a peptide consisting of EDDMMEVPY (SEQ ID NO: 1),
(b) a peptide the same as peptide (a) except that E at residue 1; E at residue 1 and D at residue 2; Y at residue 9; E at residue 1 and Y at residue 9; or E at residue 1, D at residue 2 and Y at residue 9 are deleted or substituted by an amino acid(s); or
(c) a peptide the same as peptide (a) or (b) except that M at residue 4, V at residue 7, or P at residue 8 is substituted by an amino acid.

27. A fragment of SVEP1 comprising the following peptide:
(a) a peptide consisting of EDDMMEVPY (SEQ ID NO: 1),
(b) a peptide the same as peptide (a) except that E at residue 1; E at residue 1 and D at residue 2; Y at residue 9; E at residue 1 and Y at residue 9; or E at residue 1, D at residue 2 and Y at residue 9 are deleted or substituted by an amino acid(s); or
(c) a peptide the same as peptide (a) or (b) except that M at residue 4, V at residue 7, or P at residue 8 is substituted by an amino acid,
the fragment having 2500 amino acid residues or less and an integrin-α9β1 binding activity.
